(19) United States
(12) Patent Application Publication
(10) Pub. No.: US 2026/0021079 A1
(43) Pub. Date: Jan. 22, 2026

(54) NOX INHIBITORS FOR USE IN THE TREATMENT OF ALPORT SYNDROME

(71) Applicant: CALLIDITAS THERAPEUTICS SUISSE SA, Plan-les-Ouates (CH)

(72) Inventors: Richard PHILIPSON, London (GB); Ebticem NOURI, Saint Genis Pouilly (FR); Jonathan BARRATT, Harborough (GB); Cédric SZYNDRALEWIEZ, Andilly (FR)

(21) Appl. No.: 18/863,495

(22) PCT Filed: May 9, 2023
(86) PCT No.: PCT/EP2023/062242

§ 371 Date: Nov. 6, 2024

(30) Foreign Application Priority Data
May 9, 2022 (GB) 2206759.9

(51) Int. Cl. A61K 31/437; A61K 31/401; A61K 31/403; A61K 31/55; A61K 31/675; A61P 43/00

(52) U.S. Cl. CPC A61K 31/437; A61K 31/401; A61K 31/403; A61K 31/55; A61K 31/675; A61P 43/00

(57) ABSTRACT

The present invention relates to new medical uses of NOX inhibitors. In particular, the invention relates to the use of such inhibitors in the treatment of Alport syndrome. Furthermore, the invention relates to a novel pharmaceutical combination. In particular, the invention also relates to a combination product comprising a NOX inhibitor and an ACE inhibitor.

### FIELD OF THE INVENTION

The present invention relates to new medical uses of NOX inhibitors. In particular, the invention relates to the use of such inhibitors in the treatment of Alport syndrome. Furthermore, the invention relates to a novel pharmaceutical combination.

### BACKGROUND AND PRIOR ART

Alport syndrome is a hereditary disorder of the basement membrane, resulting in a glomerulonephropathy causing renal failure. Progressive deafness and ocular anomalies may also occur (Mochizuki et al. Nat. Genet. 1994, 8, p. 77-82; Colville et al. Ophthalmic Genet. 1997, 18, p. 119-128).

Symptoms typically begin in childhood, and the first sign of the condition is usually the presence of blood in the urine (haematuria). Other symptoms of kidney disease can include having protein in the urine (proteinuria). Over time, an affected person may experience swelling (edema), bone weakening, and joint pain (osteodystrophy). Without treatment, affected individuals will experience end-stage renal disease.

Patients with Alport syndrome have been reported to show mutations in collagen IV genes (Mochizuki et al. vide supra; Lemmink et al. Hum. Mol. Genet. 1994, 3(8), p. 1269-1273 and Lemmink et al. Hum. Mutat. 1997, 9(6), p. 477-99; Gubler et al. Kidney Int. 1995, 47, p. 1142-1147). Approximately 85% of cases of Alport syndrome are X-linked and are related to mutations in the Col4a5 gene. Without treatment, 90% of males develop kidney failure by 40 years old. Females develop kidney failure less frequently and more slowly. About 14% are autosomal recessive forms that are caused by mutations in Col4a3 and Col4a4 genes, this is when both parents carry the abnormal gene and both parents pass the abnormal gene to the child. Both copies of the abnormal gene are needed to cause the autosomal recessive type of Alport Syndrome. Autosomal dominant inheritance is rare (van der Loop et al., Kidney Int. 2000, 58(5), p. 1870-1875) and occurs when one parent has the disease and passes the abnormal gene to the child.

There is no one specific treatment for Alport syndrome and the aims of current treatments include monitoring and controlling progression of the disease and treating the symptoms. Controlling patient blood pressure is very important in order to keep the kidneys as healthy as possible. Research suggests that ACE inhibitors, angiotensin receptor blockers, and statins can help reduce proteinuria and the progression of kidney disease. However, treatment of chronic kidney failure often becomes necessary, which can include dietary modifications and fluid restriction.

Although treatment may slow the progression of kidney disease in Alport syndrome, there is no cure for the disorder and no treatment has thus far been shown to completely stop kidney decline. The rate of progression of kidney decline in individuals with Alport syndrome is highly variable. In many affected individuals, kidney function eventually deteriorates to the point where dialysis or a kidney transplant is required.

There is, therefore, a real clinical need for more effective treatments of Alport syndrome and the inventors have surprisingly found that administration of a NOX inhibitor in a mouse model of Alport syndrome in collagen type IV alpha 3 chain (Col4a3) double knock out mice decreases the albumin to creatine ratio in the urine of the mice. Further studies have also shown that Blood Urea Nitrogen (BUN) and Cystatin C levels in the plasma of the double knock out mice are decreased after administration of a NOX inhibitor. These findings may render NOX inhibitors of potential use in the treatment of Alport syndrome.

The listing or discussion of an apparently prior-published document in this specification should not necessarily be taken as an acknowledgement that the document is part of the state of the art or is common general knowledge.

### DESCRIPTION OF THE INVENTION

### Medical Uses

According to a first aspect of the invention there is provided a NOX inhibitor, or a pharmaceutically acceptable salt thereof, for use in the treatment of Alport syndrome, which use is referred to hereinafter as “the use according to the invention”.

The expression “NOX inhibitor” as used herein refers to any substance that is capable of totally or partially inhibiting, blocking, attenuating, or interfering with nicotinamide adenine dinucleotide phosphate oxidase (NADPH), in particular nicotinamide adenine dinucleotide phosphate oxidase 4 (NOX4) and/or nicotinamide adenine dinucleotide phosphate oxidase 1 (NOX1). The term may be defined as the compound affects the enzymatic activity of the enzyme, the cellular localization, the stability of the protein, and/or the expression of the messenger RNA or the protein. Preferably, a NOX4/NOX1 inhibitor should be able to diminish enzyme activity and reactive oxygen species (ROS) production in a cell free assay using membranes expressing only the NOX isoform NOX4/1 protein, such as recombinant protein NOX4/1. Thus, the term “inhibitors”, when referring to NOX inhibitors, is intended to include but is not limited to, molecules, which inhibit completely or partially the activity of NOX4 and/or NOX1.

The NOX inhibitor for use in the invention may be a NOX4 inhibitor, a NOX4/1 inhibitor or a NOX1 inhibitor, or a pharmaceutically acceptable salt thereof.

For example, the NOX inhibitor may be a pyrazolopyridine NOX4 or NOX4/1 inhibitor, a pyrazolinedione NOX4 or NOX4/1 inhibitor, an amidothiazole NOX1 or NOX4/1 inhibitor, or an indole NOX4/1 inhibitor, or a pharmaceutically acceptable salt thereof. Alternatively, the NOX inhibitor may be a 2,5-disubstituted benzoxazole or a benzothiazole NOX4 inhibitor, or a pharmaceutically acceptable salt thereof.

NOX4/1 inhibitors are selective to, and thus have a major NOX inhibitory activity towards NOX4 and/or NOX1 compared to other NOX proteins, for example to NOX2 and/or NOX3/5. According to a particular embodiment, NOX4/1 inhibitors have a major NOX inhibitory activity on NOX4/1 that is higher than on other NOX proteins. For example, NOX4/1 inhibitors include small molecules, peptides, peptidomimetics, chimeric proteins, natural or unnatural proteins, nucleic acid derived polymers (such as DNA and RNA aptamers, SiRNAs, shRNAs, PNAs, or LNAs), fusion proteins with NOX4/1 antagonizing activities, antibody antagonists such as neutralizing anti-NOX4/1 antibodies, or gene therapy vectors driving the expression of such NOX4/1 antagonists.

In particular, NOX4/1 inhibitors are agents that present an inhibitory constant Ki of less than 5 micromolar in a functional ROS production assay such as those described in Gaggini et al. Bioorg. Med. Chem. 2011, 19(23), p. 6989-6999. For example, NOX4/1 inhibitors are agents that inhibit ROS production in a range of about less than 1 μM, such as between about 30 to 300 nM in a cell free assay using membrane expressing only the NOX isoform NOX4 or NOX1 protein, such as recombinant protein NOX4 or NOX1. The term “siRNA” refers to small interfering RNA, which are double stranded RNA (about 19-23 nucleotides) able to knock down or silence a targeted mRNA from a target gene. Artificial siRNAs can be either chemically synthesized as oligonucleotides or cloned into a plasmid or a virus vector (adenovirus, retrovirus or lentivirus) as short hairpin RNAs to generate a transient or stable transfection in any type of cells (Martin et al. Ann. Rev. Genomics Hum. Genet. 2007, 8, p. 81-108; Huang et al. Expert. Opin. Ther. Targets 2008, 12(5), p. 637-645).

Unless specified otherwise, relevant NOX inhibitors and pharmaceutically-acceptable salts thereof may be referred to herein as “compounds of the invention” and particular compounds are outlined in the section with the same heading below.

Pharmaceutically acceptable salts as referred to within the scope of the present invention include acid addition salts and base addition salts. Such salts may be formed by conventional means; for example, by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. under reduced pressure, by freeze-drying or by filtration). Salts may also be prepared by exchanging a counter-ion of a compound of the invention in the form of a salt with another counter-ion, for example, using a suitable ion exchange resin.

Acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxy-benzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxy-ethanesulphonate, 1- or 2-naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

Particular pharmaceutically-acceptable salts that may be mentioned include base addition salts, such as those formed with alkali metals (e.g. salts formed with Na or K), and acid addition salts, such as halide salts (e.g. chloride, bromide or iodide salts) and sulphonate salts (e.g. benzenesulphonate, methyl-, bromo- or chloro-benzenesulphonate, xylenesulphonate, methanesulphonate, ethanesulphonate, propanesulphonate, hydroxy-ethanesulphonate, 1- or 2-naphthalene-sulphonate or 1,5-naphthalenedisulphonate salts).

In an alternative aspect of the invention there is provided a method for the treatment of Alport syndrome, which method comprises the administration of an effective amount of a NOX inhibitor, or a pharmaceutically-acceptable salt thereof, to a patient in need thereof.

In a further aspect of the invention there is provided the use of a NOX inhibitor, or a pharmaceutically-acceptable salt thereof, for the manufacture of a medicament for the treatment of Alport syndrome.

Unless indicated otherwise, all technical and scientific terms used herein have the same meaning as commonly understood by one of ordinary skill in the art to which this invention pertains.

Particular features and embodiments described in relation to a given aspect of the invention should, unless the context indicates otherwise, be regarded as having been disclosed in combination with any and all other particular features and embodiments of that aspect of the invention.

Compounds of the invention are hereby indicated for use in the treatment of Alport syndrome. The skilled person will understand that references herein to the “treatment” of a condition (or, similarly, to “treating” that condition) take their normal meanings in the field of medicine. In particular, the terms may refer to achieving a reduction in the severity of one or more clinical symptom associated with the condition.

Although the term treatment may also refer herein to “prevention” of a particular condition (and, similarly, to “preventing”, or “prophylaxis” of, that condition), all of which also take their normal meanings in the art (i.e. achieving a reduction in the likelihood of developing the relevant condition or symptoms associated with the relevant condition), compounds of the invention are preferably indicated in the therapeutic treatment of Alport syndrome.

As used herein, the term “effective amount” will refer to an amount of a compound that confers the desired therapeutic effect on the treated subject (i.e. the desired treatment, as described herein). The effect may be objective (i.e. measurable by some test or marker) or subjective (i.e. the subject gives an indication of and/or feels an effect).

As used herein, references to “patients” will refer to a living subject being treated, including mammalian (in particular, human) patients, and as such “patients” may also be referred to as “subjects”, and vice versa. References to “patients” (and therefore also to “subjects”) also should be considered to refer to individuals displaying no symptoms of the relevant condition.

In a preferred aspect of the invention, the NOX inhibitor is for use in combination with an angiotensin converting enzyme (ACE) inhibitor, or a pharmaceutically-acceptable salt thereof. The inventors have surprisingly found that administration of a NOX inhibitor in combination with an ACE inhibitor in a mouse model of Alport syndrome in collagen type IV Alpha 3 chain (Col4a3) double knock out mice is more potent in decreasing the albumin to creatine ratio in the urine of the mice, with some studies pointing to a synergistic effect.

That is to say, the administration of a NOX inhibitor alone and an ACE inhibitor alone decreases the albumin to creatine ratio in the urine in the mice, but the administration of these in combination has a greater than additive effect in decreasing the albumin to creatine ratio. Furthermore, when looking at the albumin to urine levels in the mice, the results support the hypothesis the combination of the NOX inhibitor and ACE inhibitor shows a prolonged effect in maintaining proper kidney function when compared to administering the inhibitors separately to one another.

The expression “ACE inhibitor” as used herein refers to any substance that is capable of totally or partially inhibiting, blocking, attenuating, or interfering with angiotensin converting enzyme (ACE). The term may also be defined as the compound affects the enzymatic activity of the enzyme, the cellular localization, the stability of the protein, the expression of the messenger RNA or the protein.

Preferably, an ACE inhibitor should be able to diminish enzyme activity and in particular its ability to convert angiotensin I to angiotensin II. Thus, the term “inhibitors”, when referring to ACE inhibitors, is intended to include but is not limited to, molecules, which inhibit completely or partially the activity of ACE.

ACE inhibitors have a major ACE inhibitory activity component towards ACE compared to other ACE proteins. For example, ACE inhibitors include small molecules, peptides, peptidomimetics, chimeric proteins, natural or unnatural proteins, nucleic acid derived polymers (such as DNA and RNA aptamers, siRNAs, shRNAs, PNAs, or LNAs), fusion proteins with ACE antagonizing activities, antibody antagonists such as neutralizing anti-ACE antibodies, or gene therapy vectors driving the expression of such ACE antagonists.

Particular ACE inhibitors for use in combination with NOX inhibitors include ramipril, benazepril, captopril, enalapril, fosinopril, lisinopril, moexipril, perindopril, quinapril, and trandolapril. Preferably the ACE inhibitor for use in combination with NOX inhibitors is ramipril.

In another aspect of the invention, the NOX inhibitor is for use in combination with an angiotensin-receptor blocker (ARB), or a pharmaceutically-acceptable salt thereof.

In a further aspect of the invention, the NOX inhibitor is for use in combination with an ARB, or a pharmaceutically-acceptable salt thereof, and an ACE inhibitor, or a pharmaceutically-acceptable salt thereof.

The expression “angiotensin-receptor blocker (ARB)” as used herein refers to any substance that is capable of totally or partially inhibiting, blocking, attenuating, or interfering with the angiotensin II receptor type 1 (AT₁). The term may be defined as the compound affects the activity of the hormone the cellular localization, the stability of the protein, and/or the expression of the messenger RNA or the protein.

Preferably, an ARB should be able to block the activation of angiotensin II AT₁, to result in vasodilation in the patient, a reduction in the secretion of vasopressin in the patient, and/or a reduction in the production and secretion of aldosterone. Thus the term “blocker” is intended to include, but is not limited to, molecules which inhibit completely or partially the activity of angiotensin II receptor type 1.

Angiotensin-receptor blockers have a major angiotensin II receptor type 1 inhibitory activity towards angiotensin II receptor type 1 compared to other angiotensin II receptor type 1 proteins. For example, angiotensin-receptor blockers include small molecules, peptides, peptidomimetics, chimeric proteins, natural or unnatural proteins, nucleic acid derived polymers (such as DNA and RNA aptamers, siRNAs, shRNAs, PNAs, or LNAs), fusion proteins with angiotensin II receptor type 1 antagonizing activities, antibody antagonists such as neutralizing anti-angiotensin II receptor type 1 antibodies, or gene therapy vectors driving the expression of such angiotensin II receptor type 1 antagonists.

Particular angiotensin-receptor blockers for use in combination with NOX inhibitors include azilsartan, candesartan, eprosartan, irbesartan, losartan, Olmesartan, telmisartan and valsartan.

### Compounds of the Invention

### Compounds According to Formula (I)

Particular NOX inhibitors that may be included in the use of the invention include NOX inhibitors according to Formula (I):

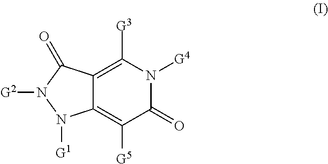

wherein:
G¹ is selected from the group consisting of:
   H;
   optionally substituted alkyl, such as aminocarbonyl alkyl (e.g. phenylacetamide);
   optionally substituted C₃-C₈-cycloalkyl alkyl;
   optionally substituted heterocycloalkyl alkyl;
   aryl alkyl, such as phenyl alkyl, for example phenyl methyl or 3-methyl phenyl methyl;
   substituted aryl alkyl, such as substituted phenyl alkyl, for example substituted phenyl methyl (e.g. 4-fluorobenzyl, 2-chlorobenzyl, 4-chlorobenzyl, 4-methyl benzyl, or 4-bromobenzyl);
   heteroaryl alkyl; and
   substituted heteroaryl alkyl, such as substituted pyridine alkyl, for example pyridine-2-yl methyl;
G² is selected from the group consisting of:
   H;
   optionally substituted alkyl;
   optionally substituted alkenyl;
   optionally substituted alkynyl;
   optionally substituted aryl, such as phenyl (e.g. 4-fluorophenyl, 4-methoxyphenyl, 4-nitrophenyl, 2-chlorophenyl, 2-methyl phenyl, 4-(trifluoromethyl)phenyl, 4-(trifluoromethoxy)phenyl, 2,5-difluorophenyl, or 2-methoxyphenyl); optionally substituted alkyl aryl;
   optionally substituted aryl alkyl;
   heteroaryl, such as benzothiazolyl, or pyridinyl;
   substituted heteroaryl, such as substituted benzothiazolyl (e.g. 1,3-benzothiazol-2-yl), or substituted pyridinyl (e.g. pyridin-2-yl);
   optionally substituted alkyl heteroaryl;
   optionally substituted heteroaryl alkyl;
   optionally substituted alkenyl aryl;
   optionally substituted aryl alkenyl;
   optionally substituted alkenyl heteroaryl;
   optionally substituted heteroaryl alkenyl;
   optionally substituted C₃-C₈-cycloalkyl;
   optionally substituted heterocycloalkyl;
   optionally substituted alkyl C₃-C₈-cycloalkyl;
   optionally substituted C₃-C₈-cycloalkyl alky;
   optionally substituted alkyl heterocycloalkyl; and
   optionally substituted heterocycloalkyl alkyl;
G³ is selected from the group consisting of:
   H;
   optionally substituted alkyl, such as optionally substituted methyl or optionally substituted ethyl;
   optionally substituted alkenyl;
   optionally substituted alkynyl;
   optionally substituted aryl, such as optionally substituted phenyl;
   optionally substituted alkyl aryl;
   optionally substituted aryl alkyl;
   optionally substituted heteroaryl;
   optionally substituted alkyl heteroaryl;
   optionally substituted heteroaryl alkyl;
   optionally substituted alkenyl aryl;
   optionally substituted aryl alkenyl;
   optionally substituted alkenyl heteroaryl;
   optionally substituted heteroaryl alkenyl;
   optionally substituted C₃-C₈-cycloalkyl;
   optionally substituted heterocycloalkyl;
   optionally substituted alkyl C₃-C₈-cycloalkyl;
   optionally substituted C₃-C₈-cycloalkyl alkyl;
   optionally substituted alkyl heterocycloalkyl; and
   optionally substituted heterocycloalkyl alkyl;
G⁴ is selected from the group consisting of:
   H;
   optionally substituted alkyl, such as optionally substituted pentyl (e.g., isopentyl);
   optionally substituted heteroalkyl;
   optionally substituted alkoxy, such as optionally substituted methoxy; (e.g. 2-methoxyethyl);
   optionally substituted alkenyl;
   optionally substituted alkynyl;
   optionally substituted aryl;
   optionally substituted alkyl aryl;
   aryl alkyl, such as benzyl or phenyl ethyl (e.g., 2-phenyl ethyl);
   substituted aryl alkyl such as substituted benzyl (e.g. benzoic acid methyl) or substituted phenyl ethyl (e.g. 4-methoxyphenyl ethyl);
   optionally substituted heteroaryl;
   optionally substituted alkyl heteroaryl;
   optionally substituted heteroaryl alkyl, such as optionally substituted thiophenyl alkyl (such as optionally substituted thiophenyl methyl, for example thiophen-2-yl methyl), optionally substituted imidazolyl alkyl (such as optionally substituted imidazolyl ethyl, for example imidazol-4-yl ethyl), optionally substituted indolyl alkyl (such as optionally substituted indolyl ethyl, for example indol-3-yl ethyl), optionally substituted furanyl alkyl (such as optionally substituted furanyl methyl, for example furan-2-yl methyl), optionally substituted benzodioxolyl alkyl (such as optionally substituted benzodioxolyl methyl, for example 1,3-benzodioxol-5-yl methyl), or optionally substituted pyridinyl methyl (such as pyridine-3-yl methyl or pyridin-2-yl methyl);
   optionally substituted alkenyl aryl;
   optionally substituted aryl alkenyl;
   optionally substituted alkenyl heteroaryl;
   optionally substituted heteroaryl alkenyl;
   optionally substituted C₃-C₈-cycloalkyl;
   heterocycloalkyl, such as morpholinyl (e.g., 5-morpholin-4-yl), piperazinyl (e.g. 4-methyl piperazinyl), or piperidinyl (e.g. 4-methylbenzyl) piperidin-4-yl);
   substituted heterocycloalkyl, such as substituted morpholinyl (e.g. 5-morpholin-4-yl), substituted piperazinyl (e.g. 4-methyl piperazinyl), or substituted piperidinyl (e.g. 4-methylbenzyl) piperidin-4-yl);
   optionally substituted alkyl C₃-C₈-cycloalkyl;
   optionally substituted C₃-C₈-cycloalkyl alkyl;
   optionally substituted alkyl heterocycloalkyl;
   heterocycloalkyl alkyl, such as morpholinyl alkyl (for example, morpholinyl ethyl, or morpholinyl propyl, in particular 2-morpholin-4-yl-ethyl, or 3-(morpholin-4-yl) propyl), piperazinyl alkyl (such as piperazinyl ethyl, for example 2-(4-acetylpiperazin-1-yl)ethyl, or 2-(4-hexanoyl piperazin-1-yl)ethyl), or pyrrolidinyl alkyl (such as pyrrolidinyl propyl, for example 3-(2-oxopyrrolidin-l-yl)propyl), or tetrahydro furanyl alkyl (such as tetrahydrofuranyl methyl, for example tetrahydrofuran-2-yl methyl); and
   substituted heterocycloalkyl alkyl such as substituted morpholinyl alkyl (for example substituted morpholinyl ethyl, or substituted morpholinyl propyl, in particular substituted 2-morpholin-4-yl-ethyl, or substituted 3-(morpholin-4-yl) propyl), substituted piperazinyl alkyl (such as substituted piperazinyl ethyl, for example substituted 2-(4-acetylpiperazin-1-yl) ethyl or substituted 2-(4-hexanoyl piperazin-1-yl)ethyl), or substituted pyrrolidinyl alkyl (such as substituted pyrrolidinyl propyl, for example substituted 3-(2-oxopyrrolidin-l-yl)propyl), or substituted tetrahydro furanyl alkyl (such as substituted tetrahydrofuranyl methyl, for example substituted tetrahydrofuran-2-yl methyl);
G⁵ is selected from the group consisting of:
   H;
   optionally substituted alkyl;
   optionally substituted alkenyl;
   optionally substituted alkynyl;
   optionally substituted aryl;
   optionally substituted alkyl aryl;
   optionally substituted aryl alkyl;
   optionally substituted heteroaryl;
   optionally substituted alkyl heteroaryl;
   optionally substituted heteroaryl alkyl;
   optionally substituted alkenyl aryl;
   optionally substituted aryl alkenyl;
   optionally substituted alkenyl heteroaryl;
   optionally substituted heteroaryl alkenyl;
   optionally substituted C₃-C₈-cycloalkyl;
   optionally substituted heterocycloalkyl;
   optionally substituted alkyl C₃-C₈-cycloalkyl;
   optionally substituted C₃-C₈-cycloalkyl alkyl;
   optionally substituted alkyl heterocycloalkyl; and
   optionally substituted heterocycloalkyl alkyl;
      as well as pharmaceutically acceptable salts thereof.

### Compounds According to Formula (II)

Further NOX inhibitors that may be included in the use of the invention include NOX inhibitors according to Formula (II):

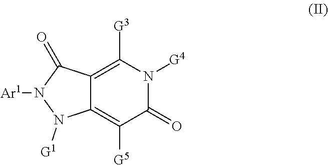

wherein:
Ar¹ is selected from the list consisting of:
   phenyl;
   phenyl substituted by a halogen, such as chloro (for example wherein Ar¹ is 2-chlorophenyl); and
   phenyl substituted by an alkoxy, such as methoxy
G¹ and G⁵ are H;
G³ is selected from the list consisting of:
   optionally substituted C₁-C₆ alkyl, such as optionally substituted methyl;
   phenyl;
   phenyl substituted by halogen, amino, alkyl amino, or alkoxy, for example wherein the phenyl is 3-chlorophenyl, 4-chlorophenyl, 2-fluorophenyl, 3-fluorophenyl, 4-fluorophenyl, 4-chloro-2-fluorophenyl, 5-chloro-2-fluorophenyl, 3-dimethylaminophenyl, 2-tri-methyl amino phenyl, 3-methyl amino phenyl, 3-amino phenyl, or 4-methoxy phenyl;
G⁴ is selected from the list consisting of:
   H;
   C₁-C₆ alkyl, such as methyl, substituted C₁-C₆ alkyl, such as substituted methyl, optionally wherein the C₁-C₆ alkyl is substituted by an alkoxy, such as wherein the substituted C₁-C₆ alkyl is methoxy ethyl, for example 2-methoxy ethyl;
   heteroaryl C₁-C₆ alkyl, such as pyridinyl C₁-C₆ alkyl (for example pyridinyl methyl, in particular pyridinyl-2-yl methyl, pyridinyl-3-yl methyl, 6-methoxypyridin-3-yl methyl, 2-methoxypyridin-4-yl methyl), or pyrazinyl C₁-C₆ alky, such as pyrazinyl-2-ylmethyl;
   substituted heteroaryl C₁-C₆ alkyl, such as substituted pyridinyl C₁-C₆ alkyl (for example substituted pyridinyl methyl, in particular substituted pyridinyl-2-yl methyl, substituted pyridinyl-3-yl methyl, substituted 6-methoxypyridin-3-yl methyl, substituted 2-methoxypyridin-4-yl methyl), or substituted pyrazinyl C₁-C₆ alky, such as substituted pyrazinyl-2-ylmethyl;
   alkoxy C₁-C₆ alkyl, such as methoxy ethyl (for example 2-methoxy ethyl); and
   substituted C₁-C₆ alkyl, such as substituted methoxy ethyl (for example substituted 2-methoxy ethyl)
      or G³ and G⁴ form together an optionally substituted 7-membered heterocycloalkyl ring comprising two nitrogen atoms, and where the two nitrogens are attached through a optionally substituted C₁-C₃ alkyl moiety,
      as well as tautomers, geometrical isomers, optically active forms and pharmaceutically acceptable salts thereof.

### Compounds According to Formula (III)

Further NOX inhibitors that may be included in the use of the invention include NOX inhibitors according to Formula (III):

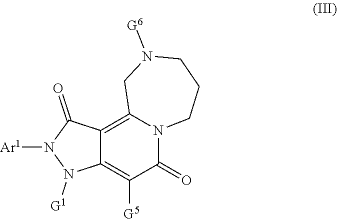

wherein:
Ar¹, G¹ and G⁵ are as defined above for compound (I) or compound (II);
G⁶ is selected from the group consisting of:
   C₁-C₆ alkyl;
   substituted C₁-C₆ alkyl;
   aryl C₁-C₆ alkyl, such as benzyl;
   substituted aryl C₁-C₆ alkyl, such as substituted phenyl C₁-C₆ alkyl, for example 2-chlorobenzyl, 3-chlorobenzyl, 4-chlorobenzyl, 2-methoxybenzyl, 3-methoxybenzyl, 4-methoxybenzyl);
   heteroaryl C₁-C₆ alkyl, such as pyridinyl C₁-C₆ alkyl, for example pyridinyl methyl (such as pyridinyl-2-yl methyl, or pyridinyl-3-yl methyl), or furanyl C₁-C₆ alkyl, for example furanyl methyl (such as furan-3-yl methyl);
   substituted heteroaryl C₁-C₆ alkyl, such as substituted pyridinyl C₁-C₆ alkyl, for example substituted pyridinyl methyl (such as substituted pyridinyl-2-yl methyl, or substituted pyridinyl-3-yl methyl), or substituted furanyl C₁-C₆ alkyl, for example substituted furanyl methyl (such as substituted furan-3-yl methyl);
      as well as tautomers, geometrical isomers, optically active forms and pharmaceutically acceptable salts and/or derivatives thereof.

### Compounds According to Formula (IV)

Alternative NOX inhibitors that may be included in the use of the invention include NOX inhibitors according to Formula (IV):

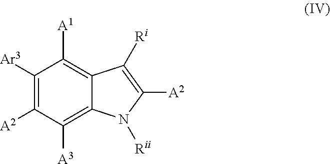

wherein:
A¹, A² and A³ are independently selected from the list consisting of:
   hydrogen;
   halogen;
   CN;
   CF₃;
   CHF₂;
   optionally substituted C₁-C₆ alkyl;
   optionally substituted C₃-C₈ cycloalkyl;
   optionally substituted heteroalkyl;
   optionally substituted heterocycloalkyl;
   optionally substituted alkoxy;
   optionally substituted amino;
   optionally substituted carboxy; and
   optionally substituted alkoxycarbonyl; and
Rⁱ is selected from the group consisting of:
   hydrogen;
   halogen;
   CN;
   CF₃;
   CHF₂;
   optionally substituted C₁-C₆ alkyl;
   optionally substituted C₃-C₈ cycloalkyl;
   optionally substituted heteroalkyl;
   optionally substituted heterocycloalkyl; and
   optionally substituted carboxy;
Rⁱⁱ is selected from the list consisting of:
   hydrogen;
   optionally substituted C₁-C₆ alkyl;
   C₃-C₈ cycloalkyl;
   optionally substituted halo C₁-C₆ alkyl;
   optionally substituted halo C₃-C₈ cycloalkyl;
   optionally substituted aryl C₁-C₆ alkyl;
   optionally substituted heteroaryl C₁-C₆ alkyl;
   optionally substituted heteroalkyl;
   optionally substituted heterocycloalkyl;
   substituted heterocycloalkyl;
   optionally substituted heterocycloalkyl C₁-C₆ alkyl;
   optionally substituted hydroxy C₁-C₆ alkyl;
   optionally substituted alkoxy C₁-C₆ alkyl;
   optionally substituted alkoxycarbonyl C₁-C₆ alkyl;
   optionally substituted aminocarbonyl C₁-C₆ alkyl;
   optionally substituted aryl C₃-C₈ cycloalkyl;
   optionally substituted heteroaryl C₃-C₈ cycloalkyl;
   optionally substituted alkoxy C₃-C₈ cycloalkyl;
   optionally substituted amino C₃-C₈ cycloalkyl; and
   optionally substituted halo C₃-C₈ cycloalkyl;
Ar³ is selected from:

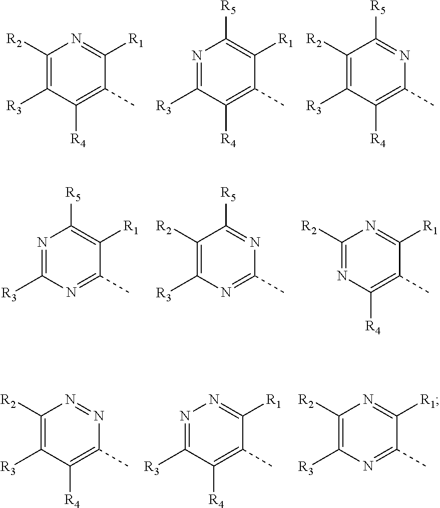

R¹ and R⁴ are independently selected from the list consisting of:
   hydrogen;
   halogen;
   CN;
   CF₃;
   CHF₂;
   NH₂;
   optionally substituted C₁-C₆ alkyl;
   optionally substituted C₃-C₈ cycloalkyl;
   alkoxy;
   amino;
   optionally substituted heterocycloalkyl;
   carboxy;
   optionally substituted alkoxy C₁-C₆ alkyl;
   optionally substituted alkoxy C₃-C₈ cycloalkyl;
   optionally substituted amino C₁-C₆ alkyl;
   optionally substituted amino C₃-C₈ cycloalkyl;
   optionally substituted amino heterocycloalkyl C₁-C₆ alkyl;
   optionally substituted carboxy C₃-C₈ cycloalkyl; and
   optionally substituted aminocarbonyl C₁-C₆ alkyl;
R² is selected from the list consisting of:
   hydrogen;
   halogen;
   CF₃;
   CHF₂;
   NH₂;
   optionally substituted C₁-C₆ alkyl;
   optionally substituted C₃-C₈ cycloalkyl;
   optionally substituted alkoxy;
   amino;
   optionally substituted heterocycloalkyl;
   carboxy;
   optionally substituted alkoxy C₁-C₆ alkyl;
   optionally substituted alkoxy C₃-C₈ cycloalkyl;
   optionally substituted amino C₁-C₆ alkyl;
   optionally substituted amino C₃-C₈ cycloalkyl;
   optionally substituted amino heterocycloalkyl C₁-C₆ alkyl;
   optionally substituted C₃-C₈ carboxy cycloalkyl; and
   optionally substituted aminocarbonyl C₁-C₆ alkyl;
R³ is selected from the list consisting of:
   hydrogen;
   halogen;
   CF₃;
   CHF₂;
   NH₂;
   optionally substituted C₁-C₆ alkyl;
   optionally substituted C₃-C₈ cycloalkyl;
   optionally substituted alkoxy;
   amino;
   optionally substituted heterocycloalkyl;
   carboxy;
   optionally substituted alkoxy C₁-C₆ alkyl;
   optionally substituted alkoxy C₃-C₈ cycloalkyl;
   optionally substituted amino C₁-C₆ alkyl;
   optionally substituted amino C₃-C₈ cycloalkyl;
   optionally substituted amino heterocycloalkyl C₁-C₆ alkyl;
   optionally substituted C₃-C₈ carboxy cycloalkyl; and
   optionally substituted aminocarbonyl C₁-C₆ alkyl;
R⁵ is selected from the group consisting of:
   hydrogen;
   halogen;
   CF₃;
   CHF₂;
   NH₂;
   optionally substituted C₁-C₆ alkyl;
   optionally substituted C₃-C₈ cycloalkyl;
   optionally substituted alkoxy;
   amino;
   optionally substituted heterocycloalkyl;
   carboxy;
   optionally substituted alkoxy C₁-C₆ alkyl;
   optionally substituted alkoxy C₃-C₈ cycloalkyl;
   optionally substituted amino C₁-C₆ alkyl;
   optionally substituted amino C₃-C₈ cycloalkyl;
   optionally substituted amino heterocycloalkyl C₁-C₆ alkyl;
   optionally substituted C₃-C₈ carboxy cycloalkyl; and
   optionally substituted aminocarbonyl C₁-C₆ alkyl,
      wherein when one from R¹, R², R³ and R⁴ is not H, the other from this group are H or any of R¹, R², R³, R⁴ and R⁵ can be linked together to form an optionally substituted bicyclic heteroaryl;
Ar³ is also selected from an optionally substituted bicyclic heteroaryl, in particular from the group consisting of:

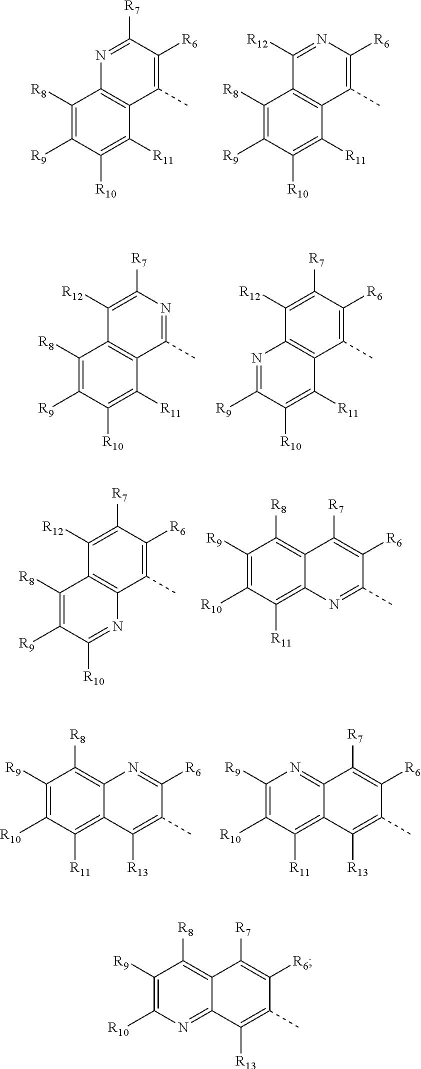

wherein R⁶, R⁷, R⁸, R⁹, R¹⁰, R¹¹, R¹² and R¹³ are each independently selected from the list consisting of:
hydrogen;
halogen;
hydroxy;
CN;
CF₃;
CHF₂;
NH₂;
alkoxy;
amino;
carboxy;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
optionally substituted alkoxy C₁-C₆ alkyl;
optionally substituted amino C₁-C₆ alkyl;
optionally substituted carboxy C₁-C₆ alkyl;
optionally substituted aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl;
optionally substituted aminocarbonyl C₃-C₈ cycloalkyl;
acylamino;
ureido;
sulfonyl; and
sulfonylamino;
   R¹⁴ is selected from the list consisting of:
hydrogen;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
optionally substituted alkoxy C₁-C₆ alkyl;
optionally substituted amino C₁-C₆ alkyl,
carboxy C₁-C₆ alkyl;
optionally substituted aminocarbonyl C₁-C₆ alkyl;
optionally substituted heterocycloalkyl C₃-C₈ cycloalkyl;
aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl, and
optionally substituted aminocarbonyl C₃-C₈ cycloalkyl;
   Ar² is selected from the following group:

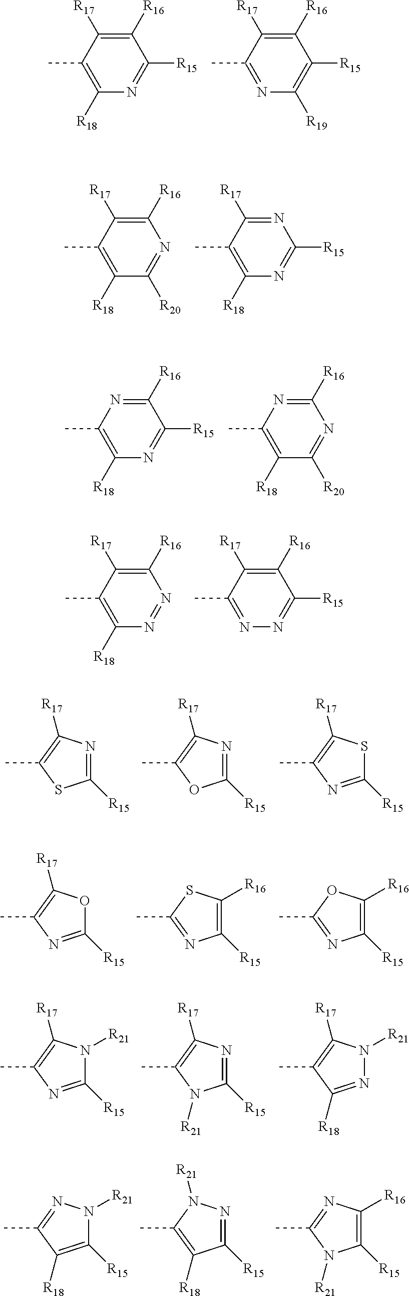

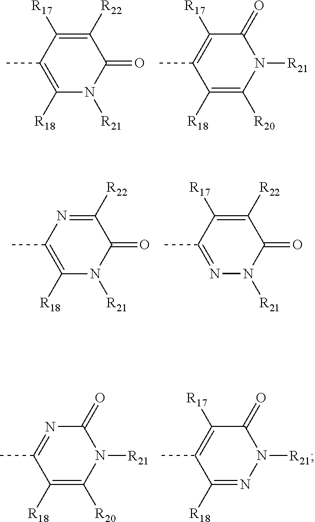

wherein R¹⁵, R¹⁶ and R¹⁹ are independently selected from the list consisting of:
hydrogen;
halogen;
hydroxy;
CN;
CF₃;
CHF₂;
NH₂;
alkoxy;
amino;
carboxy;
aminocarbonyl;
alkoxy carbonyl;
optionally substituted C₁-C₆ alkyl;
optionally substituted C₃-C₈ cycloalkyl;
optionally substituted alkenyl;
alkynyl;
optionally substituted haloalkyl;
optionally substituted heteroalkyl;
optionally substituted heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
optionally substituted alkoxy C₁-C₆ alkyl;
optionally substituted amino C₁-C₆ alkyl;
optionally substituted carboxy C₁-C₆ alkyl;
aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl;
optionally substituted aminocarbonyl C₃-C₈ cycloalkyl;
acylamino;
ureido; and
sulfonyl; or
   wherein R¹⁵, R¹⁶ and R¹⁹ may also be independently selected from the following groups:

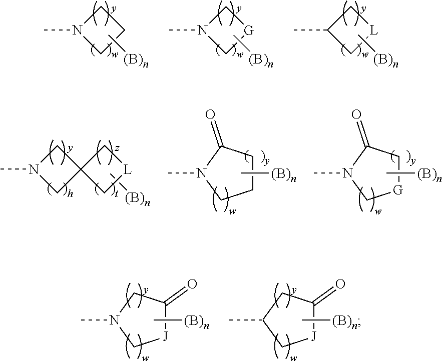

wherein y, w, z and t are independently:
an integer ranging from 1 to 3;
   wherein h is:
an integer ranging from 0 to 3;
   wherein n is:
an integer ranging from 0 to 4;
   wherein G is selected from the list consisting of:
N—R²³;
O;
S; and
SO₂;
   wherein J is selected from the list consisting of:
C—(B)n; and
N—R²³;
   wherein L is selected from the list consisting of:
C—(B)n;
N—R²³;
O;
S; and
SO₂;
   wherein R²³ is selected from the list consisting of:
hydrogen;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted aryl;
optionally substituted heteroaryl;
C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
alkoxy C₁-C₆ alkyl;
optionally substituted amino C₁-C₆ alkyl;
optionally substituted carboxy C₁-C₆ alkyl;
optionally substituted aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl;
optionally substituted aminocarbonyl C₃-C₈ cycloalkyl; and
aminosulfonyl;
   wherein B is selected from the group consisting of:
hydrogen;
halogen;
hydroxy;
CN;
CF₃;
CHF₂;
NH₂;
alkoxy;
amino;
carboxy;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
optionally substituted alkoxy C₁-C₆ alkyl;
optionally substituted amino C₁-C₆ alkyl;
optionally substituted carboxy C₁-C₆ alkyl;
aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl;
optionally substituted aminocarbonyl C₃-C₈ cycloalkyl;
acylamino;
ureido;
sulfonyl; and
sulfonylamino;
   wherein R¹⁷ and R¹⁸ are independently selected from the list consisting of:
hydrogen;
halogen;
CN;
CF₃;
CHF₂;
alkoxy;
amino;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted C₃-C₈ cycloalkyl C₁-C₆ alkyl;
optionally substituted heterocycloalkyl C₁-C₆ alkyl;
optionally substituted alkoxy C₁-C₆ alkyl;
optionally substituted amino C₁-C₆ alkyl;
optionally substituted heterocycloalkyl C₃-C₈ cycloalkyl;
optionally substituted alkoxy C₃-C₈ cycloalkyl;
optionally substituted amino C₃-C₈ cycloalkyl;
acylamino;
ureido;
sulfonyl;
aminosulfonyl; and
sulfonylamino;
   wherein R²⁰ and R²² are independently selected from the list consisting of:
hydrogen;
halogen;
CN;
CF₃;
CHF₂;
alkoxy;
amino;
optionally substituted C₁-C₆ alkyl;
optionally substituted C₃-C₈ cycloalkyl;
optionally substituted haloalkyl;
optionally substituted heteroalkyl;
optionally substituted heterocycloalkyl;
optionally substituted C₃-C₈ cycloalkyl C₁-C₆ alkyl;
optionally substituted heterocycloalkyl C₁-C₆ alkyl;
optionally substituted alkoxy C₁-C₆ alkyl;
optionally substituted amino C₁-C₆ alkyl;
optionally substituted heterocycloalkyl C₃-C₈ cycloalkyl;
optionally substituted alkoxy C₃-C₈ cycloalkyl;
and optionally substituted amino C₃-C₈ cycloalkyl;
   wherein R²¹ is selected from the list consisting of:
hydrogen;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl aryl C₁-C₆ alkyl;
optionally substituted heteroaryl C₁-C₆ alkyl;
optionally substituted alkoxy C₁-C₆ alkyl;
optionally substituted amino C₁-C₆ alkyl;
optionally substituted carboxy C₁-C₆ alkyl;
optionally substituted aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl; and
optionally substituted aminocarbonyl C₃-C₈ cycloalkyl;
   wherein R¹⁵ and R¹⁶ can be linked together to form an optionally substituted bicyclic heteroaryl having the following formula:

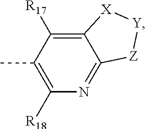

wherein X, Y and Z are each independently selected from the list consisting of:

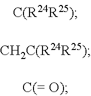

O; and
N—R²⁶;
   wherein R²⁴ and R²⁵ are each independently selected from the list consisting of:
hydrogen;
halogen;
hydroxy;
CN;
CF₃;
CHF₂;
NH₂;
alkoxy;
amino;
carboxy;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
optionally substituted alkoxy C₁-C₆ alkyl;
optionally substituted amino C₁-C₆ alkyl;
optionally substituted carboxy C₁-C₆ alkyl;
optionally substituted aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl;
optionally substituted aminocarbonyl C₃-C₈ cycloalkyl;
acylamino;
ureido;
sulfonyl; and
sulfonylamino;
   wherein R²⁴ and R²⁵ can be linked together to form an optionally substituted C₃-C₈ cycloalkyl or an optionally substituted heterocycloalkyl;
   wherein R²⁶ is selected from the group consisting of:
hydrogen;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
optionally substituted alkoxy C₁-C₆ alkyl;
amino C₁-C₆ alkyl;
optionally substituted carboxy C₁-C₆ alkyl;
optionally substituted aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl;
optionally substituted aminocarbonyl C₃-C₈ cycloalkyl; and
aminosulfonyl;
   wherein R¹⁵ and R¹⁹ can be linked together to form an optionally substituted bicyclic heteroaryl;
   wherein R¹⁵ and R²¹ can be linked together to form an optionally substituted bicyclic heteroaryl;
   wherein R¹⁷ and R²¹ can be linked together to form an optionally substituted bicyclic heteroaryl;
   wherein R²⁰ and R²¹ can be linked together to form an optionally substituted bicyclic heteroaryl;
   as well as tautomers, geometrical isomers, optically active forms, and pharmaceutically acceptable salts thereof.

In a particular embodiment, the compound according to Formula (IV) is a compound according to Formula (IVa):

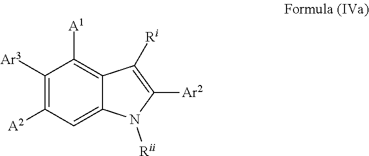

wherein Ar², Ar³, Rⁱ; Rⁱⁱ, A¹ and A²; are as defined above, wherein at least one A¹ and A² is not H.

In a particular embodiment, the compound according to Formula (IV) is a compound according to Formula (IVb):

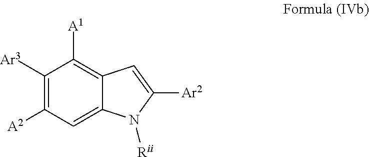

wherein Ar², Ar³, Rⁱⁱ, A¹ and A²; are as defined above.

In a particular embodiment, the compound according to Formula (IV) is a compound according to Formula (IVc):

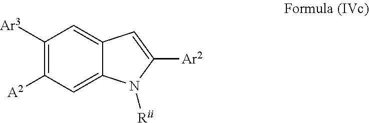

wherein Ar², Ar³, Rⁱⁱ, and A²; are as defined above.

### Compounds According to Formula (V)

Alternative NOX inhibitors that may be included in the use of the invention include NOX inhibitors according to Formula (V):

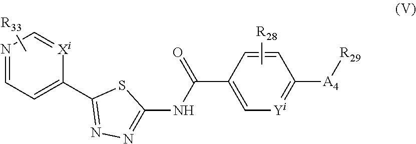

wherein:
Xⁱ is selected from the list consisting of CR²⁷ and N;
Yⁱ is selected from the list consisting of CH and N;
A₄ is selected from the list consisting of:

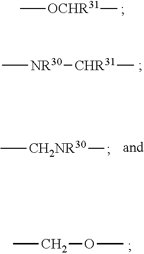

wherein R²⁷ is selected from the list consisting of:
H;
halogen;
and optionally substituted C₁-C₆ alkyl;
   wherein R²⁸ is selected from the list consisting of:
H;
halogen (e.g. chloro, fluoro);
optionally substituted alkoxy such as optionally substituted methoxy(e.g. methoxy, (tetrahydro-2H-pyran-4-yl) methoxy, piperidin-4-ylmethoxy) or optionally substituted ethoxy (e.g. 2-(dimethylamino) ethoxy, 2-hydroxy ethoxy, 1-phenyl ethoxy, 2-methoxy ethoxy);
optionally substituted alkoxy C₁-C₆ alkyl;
optionally substituted C₁-C₆ alkyl such as optionally substituted methyl;
optionally substituted amino such as optionally substituted C₁-C₆ alkyl amino (e.g. methyl amino, tetrahydro-2H-pyran-4-yl)methyl)amino, (1-methylpiperidin-4-yl)methyl)amino, di-methyl amino, optionally substituted ethyl amino such as 2-morpholino ethyl amino or 2-(dimethylamino) ethyl amino or methoxy ethyl amino, optionally substituted methyl amino such as 1-methyl-1H-imidazol-4-yl methyl amino or 2-hydroxyethyl)amino, optionally substituted propyl amino such as dimethylamino propyl amino);
optionally substituted heterocycloalkyl such as optionally substituted piperazine (e.g. methylpiperazin-1-yl);
optionally substituted C₁-C₆ alkyl heterocycloalkyl such as optionally substituted C₁-C₆ alkyl piperazine (e.g. methylpiperazin-1-yl);
optionally substituted amino C₁-C₆ alkyl;
optionally substituted alkoxy C₁-C₆ alkyl;
—O—R³⁴; and
—NR³⁵R³⁶;
   wherein R²⁹ is a group of formula —(CHR³²)n-A₅; or
R²⁹ forms with the moiety CHR³¹ from A₄ an optionally substituted ring selected from optionally substituted aryl such as an optionally substituted phenyl (e.g. phenyl or phenyl substituted by halogen such as fluoro phenyl substituted by alkoxy such as methoxy) and optionally substituted heteroaryl such as optionally substituted 1,3-dihydro-1H-indenyl (e.g. 1-(dimethylamino)-2,3-dihydro-1H-inden-2-yl, 2,3-dihydro-1H-inden-2-yl, 2,3-dihydro-1H-inden-1-yl) or optionally substituted 6,7-dihydro-5H-cyclopenta pyridinyl (e.g. 6,7-dihydro-5H-cyclopenta[b]pyridin-5-yl, 2-methylpyridin-3-yl, 5-methylpyridin-2-yl) or optionally substituted 1,2,3,4-tetrahydronaphthalenyl (e.g. 1,2,3,4-tetrahydronaphthalen-1-yl) or optionally substituted 2,3-dihydrobenzofuranyl (e.g. 2,3-dihydrobenzofuran-3-yl, 2,3-dihydro-1H-inden-1-yl) or optionally substituted thiadiazolyl (e.g. 1,3,4-thiadiazol-2-yl) or optionally substituted isoxazolyl (e.g. 5-methylisoxazol-3-yl) or optionally substituted pyrazolyl (e.g. 1-methyl-1H-pyrazol-3-yl) or optionally substituted imidazolyl (e.g. 1-methyl-1H-imidazol-2-yl); or
R²⁹ forms with the moiety NR³⁰ from A₄ an optionally substituted ring selected from the list consisting of optionally substituted aryl and optionally substituted heteroaryl such as optionally substituted isoindolinyl (e.g. isoindolin-2-yl, 1H-indol-1-yl);
   wherein n is an integer from 0 to 4 (such as 0, 1, 2, 3 or 4);
   wherein R³⁰ is selected from H and optionally substituted alkyl, such as optionally substituted methyl;
   wherein A₅ is an optionally substituted ring selected from optionally substituted aryl such as optionally substituted phenyl (e.g. methoxy phenyl, fluoro phenyl, chloro phenyl), optionally substituted heteroaryl such as optionally substituted pyridine (e.g. pyridin-2-yl, pyridin-3-yl, pyridin-4-yl, 2-methyl pyridin-3-yl, 5-methyl pyridin-2-yl) or optionally substituted pyrazolyl (e.g. 1,3-dimethyl-1H-pyrazol-5-yl, 1-methyl-1H-pyrazol-3-y) or optionally substituted thiadiazolyl (e.g. 1,3,4-thiadiazol-2-yl) or optionally substituted imidazolyl (e.g. 1H-imidazol-4-yl, 1-methyl-1H-imidazol-2-yl, 1-methyl-1H-imidazol-5-yl) or optionally substituted 1,2,4-triazolyl (e.g. 1-methyl-1H-1,2,4-triazol-5-yl) or optionally substituted isoxazolyl (e.g. 1-cyclopropylisoxazol-3-yl) or optionally substituted oxadiazolyl (e.g. 5-methyl-1,2,4-oxadiazol-3-yl) or optionally substituted pyrimidinyl (e.g. pyrimidinyl-2-yl);
   wherein R³¹ is selected from the list consisting of:
H;
optionally substituted C₁-C₆ alkyl such as optionally substituted methyl (e.g. methoxy methyl, 3,3-difluoropyrrolidin-1-yl methyl, 4-methylpiperazin-1-yl methyl, hydroxyl methyl) or optionally substituted ethyl or optionally substituted propyl (e.g. methyl, hydroxy methyl, hydroxy ethyl, 2-propanolyl, hydroxyl isopropyl);
optionally substituted amino C₁-C₆ alkyl such as optionally substituted amino methyl (e.g. dimethylamino methyl, methylamino methyl);
optionally substituted alkoxy C₁-C₆ alkyl;
optionally substituted heterocycloalkyl C₁-C₆ alkyl such as optionally substituted heterocycloalkyl methyl for example optionally substituted pyrrolidine C₁-C₆ alkyl (e.g. 3,3-difluoropyrrolidin-1-yl methyl) or substituted piperazine C₁-C₆ alkyl (e.g. 4-methylpiperazin-1-yl methyl) or heterocycloalkyl ethyl for example optionally substituted morpholino C₁-C₆ alkyl (e.g. morpholino methyl, morpholino ethyl) or optionally substituted pyrrolidine C₁-C₆ alkyl (e.g. pyrrolidine methyl, pyrrolidine ethyl);
optionally substituted aminocarbonyl (e.g. dimethyl aminocarbonyl);
optionally substituted C₂-C₈ cycloalkyl such as optionally substituted cyclopropyl; and
optionally substituted amino C₁-C₆ alkyl such as optionally substituted amino ethyl (e.g. di-methyl amino ethyl) or optionally substituted amino methyl (e.g. di-methyl amino methyl);
R³² is selected from the list consisting of:
H;
optionally substituted C₁-C₆ alkyl such as optionally substituted methyl;
optionally substituted amino;
optionally substituted C₁-C₆ alkyl amino (e.g. dimethyl amino); and
hydroxy; and
wherein R³² groups are independently selected for each repeating unit (CHR³²);
   wherein R³³ is selected from the list consisting of:
H;
halogen (e.g. fluoro); and
optionally substituted C₁-C₆ alkyl such as methyl;
   wherein R³⁴ is selected from the group consisting of:
H;
optionally substituted C₁-C₆ alkyl such as optionally substituted methyl or optionally substituted ethyl (e.g. methoxy ethyl, 2-(dimethylamino)ethyl, hydroxy ethyl);
optionally substituted amino C₁-C₆ alkyl;
optionally substituted heterocycloalkyl;
optionally substituted C₂-C₈ cycloalkyl;
optionally substituted heterocycloalkyl C₁-C₆ alkyl such as optionally substituted heterocycloalkyl methyl, for example optionally substituted tetrahydropyran C₁-C₆ alkyl (e.g. tetrahydro-2H-pyran-4-yl) or optionally substituted piperidine alkyl (e.g. 1-methylpiperidin-4-yl);
optionally substituted C₂-C₈ cycloalkyl C₁-C₆ alkyl;
optionally substituted alkoxy;
optionally substituted amino C₁-C₆ alkyl such optionally substituted amino ethyl (e.g. 2-(dimethylamino)ethyl);
optionally substituted aryl C₁-C₆ alkyl; and
optionally substituted heteroaryl C₁-C₆ alkyl;
R³⁵ and R³⁶ are independently selected from the list consisting of:
H;
optionally substituted C₁-C₆ alkyl such as optionally substituted methyl (e.g. 1-methyl-1H-imidazol-4-yl)methyl)) or optionally substituted ethyl (e.g. 2-methoxy ethyl);
optionally substituted amino C₁-C₆ alkyl such as optionally substituted amino ethyl (e.g. dimethyl amino ethyl) or such as optionally substituted amino propyl (e.g. dimethylamino)propyl);
optionally substituted heterocycloalkyl such as optionally substituted piperidine (e.g. 1-methylpiperidin);
optionally substituted C₂-C₈ cycloalkyl;
optionally substituted heterocycloalkyl C₁-C₆ alkyl such as optionally substituted heterocycloalkyl ethyl for example optionally substituted morpholino C₁-C₆ alkyl (e.g. 2-morpholino ethyl) or optionally substituted heterocycloalkyl methyl for example optionally substituted tetrahydrofuran C₁-C₆ alkyl (e.g. tetrahydro-2H-pyran-4-yl methyl) or piperidine C₁-C₆ alkyl (e.g. 1-methylpiperidin-4-yl) methyl or optionally substituted imidazolyl C₁-C₆ alkyl (e.g. 1-methyl-1H-imidazol-4-yl)methyl) optionally substituted C₂-C₈ cycloalkyl C₁-C₆ alkyl;
optionally substituted alkoxy;
optionally substituted alkoxy C₁-C₆ alkyl such as optionally substituted alkoxy ethyl (e.g. 2-methoxy ethyl);
optionally substituted aryl C₁-C₆ alkyl; and
optionally substituted heteroaryl C₁-C₆ alkyl such as heteroaryl C₁-C₆ alkyl methyl, for example optionally substituted imidazolyl C₁-C₆ alkyl (e.g. 1-methyl-1H-imidazol-4-yl methyl), optionally substituted amino C₁-C₆ alkyl such optionally substituted amino ethyl or optionally substituted amino propyl (e.g. 2-(dimethylamino)ethyl, 2-(dimethylamino)propyl)); as well as tautomers, geometrical isomers, optically active forms, pharmaceutically acceptable salts and pharmaceutically active derivative thereof.

### Further Compounds

In one aspect of the invention, the compounds of the invention may form compounds such as those described in, for example, WO 2017/192304, WO 2016/207785, WO 2019/215291, or WO 2018/203298), which are incorporated herein by reference.

### Substituent Definitions

For the avoidance of doubt, compounds of the invention may exist as solids, and thus the scope of the invention includes all amorphous, crystalline and part crystalline forms thereof. Where compounds of the invention exist in crystalline and part crystalline forms, such forms may include solvates, which are included in the scope of the invention. Compounds of the invention may also exist in solution.

The present invention also embraces isotopically-labelled compounds of the invention, which are identical, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, references to the compounds of the invention also includes deuterated compounds, i.e. in which one or more hydrogen atoms are replaced by the hydrogen isotope deuterium.

The term “alkyl” when used alone or in combination with other terms, comprises a straight chain or branched C₁-C₂₀ alkyl which refers to monovalent alkyl groups having 1 to 20 carbon atoms. This term is exemplified by groups such as methyl, ethyl, n-propyl, i-propyl, n-butyl, s-butyl, i-butyl, t-butyl, n-pentyl, 1-ethylpropyl, 2-methylbutyl, 3-methylbutyl, 2,2-dimethylpropyl, n-hexyl, 2-methylpentyl, 3-methylpentyl, 4-methylpentyl, n-heptyl, 2-methylhexyl, 3-methylhexyl, 4-methylhexyl, 5-methylhexyl, n-heptyl, n-octyl, n-nonyl, n-decyl, tetrahydrogeranyl, n-dodecyl, n-tridecyl, n-tetradecyl, n-pentadecyl, n-hexadecyl, n-octadecyl, n-nonadecyl, and n-eicosanyl. Preferably, the term “alkyl” refers to C₁-C₉ alkyl, more preferably C₁-C₆ alkyl, especially preferably C₁-C₄ alkyl, which, by analogy, refer respectively to monovalent alkyl groups having 1 to 9 carbon atoms, monovalent alkyl groups having 1 to 6 carbon atoms and monovalent alkyl groups having 1 to 4 carbon atoms.

The term “alkenyl” when used alone or in combination with other terms, comprises a straight chain or branched C₂-C₂₀ alkenyl. It may have any available number of double bonds in any available positions, and the configuration of the double bond may be the (E) or (Z) configuration. This term is exemplified by groups such as vinyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl, 5-hexenyl, 1-heptenyl, 1-octenyl, geranyl, 1-decenyl, 1-tetradecenyl, 1-octadecenyl, 9-octadecenyl, 1-eicosenyl, and 3,7,11,15-tetramethyl-l-hexadecenyl, and the like. Preferably, the term “alkenyl” refers to C₂-C₈ alkenyl, more preferably C₂-C₆ alkenyl. Especially preferred are vinyl or ethenyl (—CH≡CH₂), n-2-propenyl (allyl, —CH₂CH≡CH₂), isopropenyl, 1-propenyl, 2-methyl-1-propenyl, 1-butenyl, 2-butenyl, and 3-methyl-2-butenyl.

The term “alkynyl” when used alone or in combination with other terms, comprises a straight chain or branched C₂-C₂₀ alkynyl. It may have any available number of triple bonds in any available positions. This term is exemplified by groups such as alkynyl groups that may have a carbon number of 2-20, and optionally a double bond, such as ethynyl (—C≡CH), 1-propynyl, 2-propynyl (propargyl: —CH₂C≡CH), 2-butynyl, and 2-pentene-4-ynyl. Particularly, these include C₂-C₈ alkynyl, and more preferably C₂-C₆ alkynyl. Preferably those include C₂-C₆ alkynyl which refers to groups having 2 to 6 carbon atoms and having at least 1 or 2 sites of alkynyl unsaturation.

The term “heteroalkyl” refers to C₁-C₁₂-alkyl, preferably C₁-C₆-alkyl, wherein at least one carbon has been replaced by a heteroatom selected from O, N or S. A particularly preferable heteroalkyl is 2-methoxy ethyl.

The term “aryl” refers to an unsaturated aromatic carbocyclic group of from 6 to 14 carbon atoms having a single ring (e.g. phenyl) or multiple condensed rings (e.g., indenyl, naphthyl) and the term includes phenyl, naphthyl, anthryl, and phenanthrenyl.

The term “C₁-C₆ alkyl aryl” refers to aryl groups having a C₁-C₆ alkyl substituent, including methyl phenyl, and ethyl phenyl.

The term “aryl C₁-C₆ alkyl” refers to C₁-C₆ alkyl groups having an aryl substituent, including 3-phenylpropanyl, and benzyl.

The term “heteroaryl” refers to a monocyclic heteroaromatic, or a bicyclic or a tricyclic fused-ring heteroaromatic group. Particular examples of heteroaromatic groups include optionally substituted pyridyl, pyrrolyl, pyrimidinyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, and benzoquinolyl.

The term “C₁-C₆ alkyl heteroaryl” refers to heteroaryl groups having a C₁-C₆ alkyl substituent, including methyl furyl.

The term “heteroaryl C₁-C₆ alkyl” refers to C₁-C₆ alkyl groups having a heteroaryl substituent, including furyl methyl.

The term “C₂-C₆ alkenyl aryl” refers to an aryl groups having a C₂-C₆ alkenyl substituent, including vinyl phenyl.

The term “aryl C₂-C₆ alkenyl” refers to a C₂-C₆ alkenyl groups having an aryl substituent, including phenyl vinyl.

The term “C₂-C₆ alkenyl heteroaryl” refers to heteroaryl groups having a C₂-C₆ alkenyl substituent, including vinyl pyridinyl.

The term “heteroaryl C₂-C₆ alkenyl” refers to C₂-C₆ alkenyl groups having a heteroaryl substituent, including pyridinyl vinyl.

The term “C₃-C₈ cycloalkyl” refers to a saturated carbocyclic group of from 3 to 8 carbon atoms having a single ring (e.g. cyclohexyl) or multiple condensed rings (e.g., norbornyl). C₃-C₈ cycloalkyl includes cyclopentyl, cyclohexyl, and norbornyl.

The term “heterocycloalkyl” refers to a C₃-C₈ cycloalkyl group according to the definition above, in which up to 3 carbon atoms are replaced by heteroatoms chosen from the group consisting of O, S, NR, R being defined as hydrogen or methyl. The term “heterocycloalkyl” includes azetidinyl, pyrrolidinyl, piperidinyl, piperazinyl, morpholinyl, and tetrahydrofuranyl.

The term “C₁-C₆ alkyl C₃-C₈ cycloalkyl” refers to C₃-C₈ cycloalkyl groups having a C₁-C₆ alkyl substituent, including methyl cyclopentyl.

The term “C₃-C₈ cycloalkyl C₁-C₆ alkyl” refers to C₁-C₆ alkyl groups having a C₃-C₈-cycloalkyl substituent, including 3-cyclopentyl propyl.

The term “C₁-C₆ alkyl heterocycloalkyl” refers to heterocycloalkyl groups having a C₁-C₆ alkyl substituent, including 4-methylpiperidinyl.

The term “heterocycloalkyl C₁-C₆ alkyl” refers to C₁-C₆ alkyl groups having a heterocycloalkyl substituent, including (1-methylpiperidin-4-yl)methyl.

The term “carboxy” refers to the group —C(O)OH.

The term “carboxy C₁-C₆ alkyl” refers to C₁-C₆ alkyl groups having a carboxy substituent, including 2-carboxyethyl.

The term “acyl” refers to the group —C(O)R where R is selected from the list consisting of H, C₁-C₆ alkyl, aryl, heteroaryl, C₃-C₈-cycloalkyl, heterocycloalkyl, aryl C₁-C₆ alkyl, heteroaryl C₁-C₆ alkyl, C₃-C₈ cycloalkyl C₁-C₆ alkyl and heterocycloalkyl C₁-C₆ alkyl, including acetyl. Preferably R is C₁-C₆ alkyl.

The term “acyl C₁-C₆ alkyl” refers to C₁-C₆ alkyl groups having an acyl substituent, including 2-acetylethyl.

The term “acyl aryl” refers to aryl groups having an acyl substituent, including 2-acetylphenyl.

The term “acyloxy” refers to the group —OC(O)R where R is selected from the list consisting of H, C₁-C₆ alkyl, C₂-C₆ alkenyl, C₂-C₆ alkynyl, C₃-C₈-cycloalkyl, heterocycloalkyl, aryl, heteroaryl, aryl C₁-C₆ alkyl, heteroaryl C₁-C₆ alkyl, aryl C₂-C₆ alkenyl, heteroaryl C₂-C₆ alkenyl, aryl C₂-C₆ alkynyl, heteroaryl C₂-C₆ alkynyl, C₃-C₈-cycloalkyl C₁-C₆ alkyl, and heterocycloalkyl C₁-C₆ alkyl, including acetyloxy.

The term “acyloxy C₁-C₆ alkyl” refers to C₁-C₆ alkyl groups having an acyloxy substituent, including 2-(ethylcarbonyloxy) ethyl.

The term “alkoxy” refers to the group —O—R where R is selected from the list consisting of C₁-C₆ alkyl, aryl, heteroaryl, aryl C₁-C₆ alkyl and heteroaryl C₁-C₆ alkyl. Preferred alkoxy groups include for example, methoxy, ethoxy, and phenoxy.

The term “alkoxy C₁-C₆ alkyl” refers to C₁-C₆ alkyl groups having an alkoxy substituent, including methoxyethyl.

The term “alkoxycarbonyl” refers to the group —C(O)OR where R includes C₁-C₆ alkyl, aryl, heteroaryl, aryl C₁-C₆ alkyl, heteroaryl C₁-C₆ alkyl and heteroalkyl.

The term “alkoxycarbonyl C₁-C₆ alkyl” refers to C₁-C₆ alkyl groups having an alkoxycarbonyl substituent, including 2-(benzyloxycarbonyl)ethyl.

The term “aminocarbonyl” refers to the group —C(O)NRR′ where R and R′ are independently selected from the list consisting of H, C₁-C₆ alkyl, aryl, heteroaryl, aryl C₁-C₆ alkyl and heteroaryl C₁-C₆ alkyl, including N-phenyl carbonyl.

The term “aminocarbonyl C₁-C₆ alkyl” refers to alkyl groups having an aminocarbonyl substituent, including 2-(dimethylaminocarbonyl)ethyl, N-ethyl acetamidyl, and N,N-Diethyl-acetamidyl.

The term “acylamino” refers to the group —NRC(O)R′ where R and R′ are independently selected from the list consisting of H, C₁-C₆ alkyl, C₂-C₆ alkenyl, C₂-C₆ alkynyl, C₃-C₈-cycloalkyl, heterocycloalkyl, aryl, heteroaryl, aryl C₁-C₆ alkyl, heteroaryl C₁-C₆ alkyl, aryl C₂-C₆ alkenyl, heteroaryl C₂-C₆ alkenyl, aryl C₂-C₆ alkynyl, heteroaryl C₂-C₆ alkynyl, cycloalkyl C₁-C₆ alkyl, and heterocycloalkyl C₁-C₆ alkyl, including acetylamino.

The term “acylamino C₁-C₆ alkyl” refers to C₁-C₆ alkyl groups having an acylamino substituent, including 2-(propionylamino) ethyl.

The term “ureido” refers to the group —NRC(O)NR′R″ where R, R′ and R″ are independently selected from the list consisting of H, C₁-C₆ alkyl, C₂-C₆ alkenyl, alkynyl, C₃-C₈-cycloalkyl, heterocycloalkyl, aryl, heteroaryl, aryl C₁-C₆ alkyl, heteroaryl C₁-C₆ alkyl, aryl C₂-C₆ alkenyl, heteroaryl C₂-C₆ alkenyl, aryl C₂-C₆ alkynyl, heteroaryl C₂-C₆ alkynyl, cycloalkyl C₁-C₆ alkyl, and heterocycloalkyl C₁-C₆ alkyl, and where R′ and R″, together with the nitrogen atom to which they are attached, can optionally form a 3-8-membered heterocycloalkyl ring.

The term “ureido C₁-C₆ alkyl” refers to C₁-C₆ alkyl groups having an ureido substituent, including 2-(N′-methylureido) ethyl.

The term “carbamate” refers to the group —NRC(O)OR′ where R and R′ are independently selected from the list consisting of C₁-C₆ alkyl, C₂-C₆ alkenyl, C₂-C₆ alkynyl, C₃-C₈-cycloalkyl, heterocycloalkyl, aryl, heteroaryl, C₁-C₆ alkyl aryl, heteroaryl C₁-C₆ alkyl, aryl C₂-C₆ alkenyl, heteroaryl C₂-C₆ alkenyl, aryl C₂-C₆ alkynyl, heteroaryl C₂-C₆ alkynyl, cycloalkyl C₁-C₆ alkyl, and heterocycloalkyl C₁-C₆ alkyl, and wherein R may also be hydrogen.

The term “amino” refers to the group —NRR′ where R and R′ are independently selected from the list consisting of H, C₁-C₆ alkyl, aryl, heteroaryl, C₁-C₆ alkyl aryl, C₁-C₆ alkyl heteroaryl, cycloalkyl, and heterocycloalkyl, and where R and R′, together with the nitrogen atom to which they are attached, can optionally form a 3-8-membered heterocycloalkyl ring.

The term “amino alkyl” refers to alkyl groups having an amino substituent, including 2-(1-pyrrolidinyl)ethyl.

The term “ammonium” refers to a positively charged group —N⁺RR′R″ where R, R′ and R″ are independently selected from the list consisting of C₁-C₆ alkyl, C₁-C₆ alkyl aryl, C₁-C₆ alkyl heteroaryl, cycloalkyl, and heterocycloalkyl, and where R and R′, together with the nitrogen atom to which they are attached, can optionally form a 3-8-membered heterocycloalkyl ring.

The term “ammonium alkyl” refers to alkyl groups having an ammonium substituent, including 1-ethylpyrrolidinium.

The term “halogen” refers to fluoro, chloro, bromo and iodo atoms.

The term “sulfonyloxy” refers to a group —OSO₂—R wherein R is selected from the group consisting of C₁-C₆ alkyl, C₁-C₆ alkyl substituted with halogens, e.g., an —OSO₂—CF₃ group, C₂-C₆ alkenyl, alkynyl, C₃-C₈-cycloalkyl, heterocycloalkyl, aryl, heteroaryl, aryl C₁-C₆ alkyl, heteroaryl C₁-C₆ alkyl, aryl C₂-C₆ alkenyl, heteroaryl C₂-C₆ alkenyl, aryl C₂-C₆ alkynyl, heteroaryl C₂-C₆ alkynyl, cycloalkyl C₁-C₆ alkyl, and heterocycloalkyl alkyl.

The term “sulfonyloxy C₁-C₆ alkyl” refers to alkyl groups having a sulfonyloxy substituent, including 2-(methylsulfonyloxy) ethyl.

The term “sulfonyl” refers to group “—SO₂—R” wherein R is selected from the group consisting of aryl, heteroaryl, C₁-C₆ alkyl, C₁-C₆ alkyl substituted with halogens (e.g., an —SO₂—CF₃ group), C₂-C₆ alkenyl, C₂-C₆ alkynyl, C₃-C₈-cycloalkyl, heterocycloalkyl, aryl, heteroaryl, aryl C₁-C₆ alkyl, heteroaryl C₁-C₆ alkyl, aryl C₂-C₆ alkenyl, heteroaryl C₂-C₆ alkenyl, aryl C₂-C₆ alkynyl, heteroaryl C₂-C₆ alkynyl, cycloalkyl C₁-C₆ alkyl, and heterocycloalkyl C₁-C₆ alkyl.

The term “sulfonyl C₁-C₆ alkyl” refers to alkyl groups having a sulfonyl substituent, including 2-(methylsulfonyl)ethyl.

The term “sulfinyl” refers to a group “—S(O)—R” wherein R is selected from the group consisting of alkyl, alkyl substituted with halogens (e.g., a —SO—CF₃ group), C₂-C₆ alkenyl, C₂-C₆ alkynyl, C₃-C₈-cycloalkyl, heterocycloalkyl, aryl, heteroaryl, aryl C₁-C₆ alkyl, heteroaryl C₁-C₆ alkyl, aryl C₂-C₆ alkenyl, heteroaryl C₂-C₆ alkenyl, aryl C₂-C₆ alkynyl, heteroaryl C₂-C₆ alkynyl, C₃-C₈-cycloalkyl C₁-C₆ alkyl, and heterocycloalkyl C₁-C₆ alkyl.

The term “sulfinyl alkyl” refers to alkyl groups having a sulfinyl substituent, including 2-(methylsulfinyl)ethyl.

The term “sulfanyl” refers to groups —S—R where R is selected from the group consisting of H, C₁-C₆ alkyl, C₁-C₆ alkyl substituted with halogen (e.g., a —S—CF₃ group), C₂-C₆ alkenyl, C₂-C₆ alkynyl, C₃-C₈-cycloalkyl, heterocycloalkyl, aryl, heteroaryl, aryl C₁-C₆ alkyl, heteroaryl C₁-C₆ alkyl, aryl C₂-C₆ alkenyl, heteroaryl C₂-C₆ alkenyl, aryl C₂-C₆ alkynyl, alkynylheteroaryl, cycloalkyl C₁-C₆ alkyl, and heterocycloalkyl C₁-C₆ alkyl. Preferred sulfanyl groups include methylsulfanyl, and ethylsulfanyl.

The term “sulfanyl C₁-C₆ alkyl” refers to C₁-C₅-alkyl groups having a sulfanyl substituent, including 2-(ethylsulfanyl)ethyl.

The term “sulfonylamino” refers to a group —NRSO₂—R′ where R and R′ are independently selected from the group consisting of C₁-C₆ alkyl, C₂-C₆ alkenyl, C₂-C₆ alkynyl, C₃-C₈-cycloalkyl, heterocycloalkyl, aryl, heteroaryl, aryl C₁-C₆ alkyl, heteroaryl C₁-C₆ alkyl, aryl C₂-C₆ alkenyl, heteroaryl C₂-C₆ alkenyl, aryl C₂-C₆ alkynyl, heteroaryl C₂-C₆ alkynyl, C₃-C₈-cycloalkyl C₁-C₆ alkyl, and heterocycloalkyl C₁-C₆ alkyl.

The term “sulfonylamino C₁-C₆ alkyl” refers to alkyl groups having a sulfonylamino substituent, including 2-(ethylsulfonylamino) ethyl.

The term “aminosulfonyl” refers to a group —SO₂—NRR′ where R and R′ are independently selected from the group consisting of H, C₁-C₆ alkyl, C₂-C₆ alkenyl, C₂-C₆ alkynyl, C₃-C₈-cycloalkyl, heterocycloalkyl, aryl, heteroaryl, aryl C₁-C₆ alkyl, heteroaryl C₁-C₆ alkyl, aryl alkenyl, heteroaryl C₂-C₆ alkenyl, aryl C₂-C₆ alkynyl, heteroaryl C₂-C₆ alkynyl, C₃-C₈-cycloalkyl C₁-C₆ alkyl, and heterocycloalkyl C₁-C₆ alkyl, and where R and R′, together with the nitrogen atom to which they are attached, can optionally form a 3-8-membered heterocycloalkyl ring. Aminosulfonyl groups include cyclohexylaminosulfonyl, and piperidinylsulfonyl.

The term “aminosulfonyl C₁-C₆ alkyl” refers to C₁-C₆ alkyl groups having an aminosulfonyl substituent, including 2-(cyclohexylaminosulfonyl)ethyl.

Unless otherwise constrained by the definition of the individual substituent, all the above substituents should be understood as being all optionally substituted.

Unless otherwise constrained by the definition of the individual substituent, the term “substituted” refers to groups substituted with from 1 to 5 substituents selected from the group consisting of C₁-C₆ alkyl, C₂-C₆ alkenyl, C₂-C₆ alkynyl, C₃-C₈ cycloalkyl, heterocycloalkyl, C₁-C₆ alkyl aryl, C₁-C₆ alkyl heteroaryl, C₁-C₆ alkyl cycloalkyl, C₁-C₆ alkyl heterocycloalkyl, cycloalkyl C₁-C₆ alkyl, heterocycloalkyl C₁-C₆ alkyl, amino, alkyl amino, aminosulfonyl, ammonium, alkoxy, acyl, acyl amino, amino carbonyl, aryl, aryl C₁-C₆ alkyl, heteroaryl, heteroaryl C₁-C₆ alkyl, sulfinyl, sulfonyl, sulphonamide, alkoxy, alkoxy carbonyl, carbamate, sulfanyl, halogen, carboxy, trihalomethyl, cyano, hydroxy, mercapto, and nitro.

Particular NOX inhibitors that may be used in the use of the invention include those selected from the following group consisting of:
2-(2-chlorophenyl)-4-methyl-5-(pyridin-2-ylmethyl)-1H-pyrazolo[4,3-c]pyridine-3,6(2H,5H)-dione;
2-(2-chlorophenyl)-4-[3-(dimethylamino)phenyl]-5-methyl-1H-pyrazolo[4,3-c]pyridine-3,6(2H,5H)-dione;
4-(2-fluoro-4-methoxyphenyl)-2-(2-methoxyphenyl)-5-(pyridin-3-ylmethyl)-1H-pyrazolo[4,3-c]pyridine-3,6(2H,5H)-dione;
(R)-3-methoxy-4-(2-morpholino-1-phenylethoxy)-N-(5-(pyridin-4-yl)-1,3,4-thiadiazol-2-yl)benzamide;
10-benzyl-2-(2-chlorophenyl)-2,3,8,9,10,11-hexahydro-1H-pyrazolo[4′,3′: 3,4]pyrido[1,2-a][1,4]diazepine-1,5(7H)-dione;
(S)-3-methoxy-4-(1-phenylethoxy)-N-(5-(pyridin-4-yl)-1,3,4-thiadiazol-2-yl)benzamide;
(R)-4-(2-hydroxy-1-phenylethoxy)-3-methoxy-N-(5-(pyridin-4-yl)-1,3,4-thiadiazol-2-yl)benzamide; and
(R)-4-(2-(dimethylamino)-1-phenylethoxy)-3-methoxy-N-(5-(pyridin-4-yl)-1,3,4-thiadiazol-2-yl)benzamide.

### Particular Compounds of the Invention

Further particular NOX inhibitors that may be used in the use of the invention include those selected from the following group consisting of:
5-(2-methoxypyridin-4-yl)-2-(2-methylpyridin-4-yl)-1H-indole;
2,5-bis(2-methylpyridin-4-yl)-1H-indole;
4-(2-(2-methylpyridin-4-yl)-1H-indol-5-yl)pyridin-2-amine;
5-(5-fluoropyridin-3-yl)-2-(2-methylpyridin-4-yl)-1H-indole;
5-(5-chloropyridin-3-yl)-2-(2-methylpyridin-4-yl)-1H-indole;
5-(5-isopropoxypyridin-3-yl)-2-(2-methylpyridin-4-yl)-1H-indole;
N,N-dimethyl-5-(2-(2-methylpyridin-4-yl)-1H-indol-5-yl)pyridin-3-amine;
5-(6-methylpyridin-3-yl)-2-(2-methylpyridin-4-yl)-1H-indole;
2-(2-methylpyridin-4-yl)-5-(pyrimidin-5-yl)-1H-indole;
2-methyl-5-(2-(2-methylpyridin-4-yl)-1H-indol-5-yl)-1H-benzo[d]imidazole;
2-(2-(azetidin-1-yl)pyridin-4-yl)-5-(5-methoxypyridin-3-yl)-1H-indole;
5-(5-methoxypyridin-3-yl)-2-(2-methoxypyridin-4-yl)-1H-indole;
2-(2,6-dimethylpyridin-4-yl)-5-(5-methoxypyridin-3-yl)-1H-indole;
5-(5-methoxypyridin-3-yl)-2-(2-methylpyrimidin-4-yl)-1H-indole;
5-(5-methoxypyridin-3-yl)-2-(1-methyl-1H-imidazol-5-yl)-1H-indole;
4-(5-(5-methoxypyridin-3-yl)-1H-indol-2-yl)-2-methylthiazole;
2-(2-cyclopropylpyridin-4-yl)-5-(5-methoxypyridin-3-yl)-1H-indole;
1-(4-(5-(5-methoxypyridin-3-yl)-1H-indol-2-yl)pyridin-2-yl)azetidin-3-ol;
1-(4-(5-(5-methoxypyridin-3-yl)-1H-indol-2-yl)pyridin-2-yl)-6-oxa-1-azaspiro[3.3]heptane;
2-(2-(4,4-difluoropiperidin-1-yl)pyridin-4-yl)-5-(5-methoxypyridin-3-yl)-1H-indole;
4-(4-(5-(5-methoxypyridin-3-yl)-1H-indol-2-yl)pyridin-2-yl)morpholine;
2-(5-fluoropyridin-3-yl)-5-(5-methoxypyridin-3-yl)-1H-indole;
5-(5-methoxypyridin-3-yl)-2-(6-methoxypyridin-3-yl)-1H-indole;
4-(5-(5-(5-methoxypyridin-3-yl)-1H-indol-2-yl)pyridin-2-yl)morpholine;
5-(5-methoxypyridin-3-yl)-2-(6-(oxetan-3-yl)pyridin-3-yl)-1H-indole;
1-(5-(5-(5-methoxypyridin-3-yl)-1H-indol-2-yl)pyridin-2-yl)-6-oxa-1-azaspiro[3.3]heptane;
N,N-dimethyl-5-(5-(2-methylpyridin-4-yl)-1H-indol-2-yl)pyridin-2-amine;
4-(5-(5-methoxypyridin-3-yl)-1H-indol-2-yl)-N,N-dimethylpyridin-2-amine;
N,N-dimethyl-4-(5-(quinolin-4-yl)-1H-indol-2-yl)pyridin-2-amine;
1-(5-(6-chloro-5-(5-methoxypyridin-3-yl)-1H-indol-2-yl)pyridin-2-yl)-6-oxa-1-azaspiro[3.3]heptane;
1-(4-(6-chloro-5-(5-methoxypyridin-3-yl)-1H-indol-2-yl)pyridin-2-yl)-6-oxa-1-azaspiro[3.3]heptane;
1-(4-(6-chloro-5-(2-methylpyridin-4-yl)-1H-indol-2-yl)pyridin-2-yl)-6-oxa-1-azaspiro [3.3]heptane;
6-(5-(6-chloro-5-(5-methoxypyridin-3-yl)-1H-indol-2-yl)pyridin-2-yl)-2-oxa-6-azaspiro[3.3]heptane;
2-(5-(6-chloro-5-(5-methoxypyridin-3-yl)-1H-indol-2-yl)pyridin-2-yl)-7-oxa-2-azaspiro[3.5]nonane;
1-(5-(6-chloro-5-(5-methoxypyridin-3-yl)-1H-indol-2-yl)pyridin-2-yl)-3-methylazetidin-3-ol;
6-chloro-2-(6-(3,3-difluoroazetidin-1-yl)pyridin-3-yl)-5-(5-methoxypyridin-3-yl)-1H-indole;
1-(5-(6-chloro-5-(5-methoxypyridin-3-yl)-1H-indol-2-yl)pyridin-2-yl)azetidine-3-carboxylic acid;
1-(5-(6-chloro-5-(5-methoxypyridin-3-yl)-1H-indol-2-yl)pyridin-2-yl)azetidine-3-carboxamide;
(1-(5-(6-chloro-5-(5-methoxypyridin-3-yl)-1H-indol-2-yl)pyridin-2-yl)azetidin-3-yl)methanol;
6-chloro-5-(5-methoxypyridin-3-yl)-2-(6-(3-methoxypyrrolidin-1-yl)pyridin-3-yl)-1H-indole;
N-(1-(5-(6-chloro-5-(5-methoxypyridin-3-yl)-1H-indol-2-yl)pyridin-2-yl)pyrrolidin-3-yl)methanesulfonamide;
(1-(5-(6-chloro-5-(5-methoxypyridin-3-yl)-1H-indol-2-yl)pyridin-2-yl)pyrrolidin-2-yl)methanol;
1-(5-(6-chloro-5-(5-methoxypyridin-3-yl)-1H-indol-2-yl)pyridin-2-yl)-5-(hydroxymethyl)pyrrolidin-2-one;
4-(5-(6-chloro-5-(5-methoxypyridin-3-yl)-1H-indol-2-yl)-3-fluoropyridin-2-yl) morpholine;
6-chloro-5-(5-methoxypyridin-3-yl)-2-(6-(4-(oxetan-3-yl) piperazin-1-yl)pyridin-3-yl)-1H-indole;
6-chloro-2-(6-(4-ethylpiperazin-1-yl)pyridin-3-yl)-5-(5-methoxypyridin-3-yl)-1H-indole 4-(5-(6-chloro-5-(5-methoxypyridin-3-yl)-1H-indol-2-yl)pyridin-2-yl)thiomorpholine 1,1-dioxide;
6-chloro-2-(6-(4,4-difluoropiperidin-1-yl)pyridin-3-yl)-5-(5-methoxypyridin-3-yl)-1H-indole;
4-(5-(6-chloro-5-(5-methoxypyridin-3-yl)-1H-indol-2-yl)pyridin-2-yl)-1-methylpiperazin-2-one;
3′-(6-chloro-5-(5-methoxypyridin-3-yl)-1H-indol-2-yl)-1-methyl-5′H-spiro[azetidine-3,7′-furo[3,4-b]pyridine];
7-(6-chloro-5-(5-methoxypyridin-3-yl)-1H-indol-2-yl)-4-methyl-3,4-dihydro-2H-pyrido[3,2-b][1,4]oxazine;
(5-(6-chloro-5-(5-methoxypyridin-3-yl)-1H-indol-2-yl)pyridin-2-yl) (oxetan-3-yl) methanol;
6-chloro-2-(6-(methoxy(oxetan-3-yl)methyl)pyridin-3-yl)-5-(5-methoxypyridin-3-yl)-1H-indole;
2-(5-(6-chloro-5-(5-methoxypyridin-3-yl)-1H-indol-2-yl)pyridin-2-yl) propan-1-ol;
5-(6-chloro-5-(5-methoxypyridin-3-yl)-1H-indol-2-yl)-1-methylpyridin-2(1H)-one;
4-(6-chloro-5-(5-methoxypyridin-3-yl)-1H-indol-2-yl)-1-cyclopropylmethyl)pyridin-2(1H)-one;
4-(5-(7-chloro-5-(5-methoxypyridin-3-yl)-1H-indol-2-yl)pyridin-2-yl)morpholine;
4-(5-(4,6-difluoro-5-(5-methoxypyridin-3-yl)-1H-indol-2-yl)pyridin-2-yl)morpholine;
4-(5-(6-chloro-5-(quinolin-5-yl)-1H-indol-2-yl)pyridin-2-yl)morpholine;
4-(5-(6-chloro-5-(5-chloropyridin-3-yl)-1H-indol-2-yl)pyridin-2-yl)morpholine;
4-fluoro-5-(5-methoxypyridin-3-yl)-2-(2-methylpyridin-4-yl)-1H-indole;
6-fluoro-5-(5-methoxypyridin-3-yl)-2-(2-methylpyridin-4-yl)-1H-indole;
4-chloro-5-(5-methoxypyridin-3-yl)-2-(2-methylpyridin-4-yl)-1H-indole;
6-chloro-5-(5-methoxypyridin-3-yl)-2-(2-methylpyridin-4-yl)-1H-indole;
5-(5-methoxypyridin-3-yl)-4-methyl-2-(2-methylpyridin-4-yl)-1H-indole;
5-(5-methoxypyridin-3-yl)-6-methyl-2-(2-methylpyridin-4-yl)-1H-indole;
4-methyl-2,5-di(pyridin-4-yl)-1H-indole;
6-methyl-2,5-di(pyridin-4-yl)-1H-indole;
4-(5-(4-chloro-5-(5-methoxypyridin-3-yl)-1H-indol-2-yl)pyridin-2-yl)morpholine;
4-(5-(6-chloro-5-(5-methoxypyridin-3-yl)-1H-indol-2-yl)pyridin-2-yl)morpholine;
4-chloro-2,5-di(pyridin-4-yl)-1H-indole
6-chloro-2,5-di(pyridin-4-yl)-1H-indole;
N,N-dimethyl-4-(2-(2-methylpyridin-4-yl)-1H-indol-5-yl)pyridin-2-amine;
5-(6-cyclopropylpyridin-3-yl)-2-(2-methylpyridin-4-yl)-1H-indole;
5-(6-methoxypyridin-3-yl)-2-(2-methylpyridin-4-yl)-1H-indole;
5-(6-(methoxymethyl)pyridin-3-yl)-2-(2-methylpyridin-4-yl)-1H-indole;
5-(5-ethoxypyridin-3-yl)-2-(2-methylpyridin-4-yl)-1H-indole;
5-(5-cyclopropoxypyridin-3-yl)-2-(2-methylpyridin-4-yl)-1H-indole;
5-(5-(cyclopropylmethoxy)pyridin-3-yl)-2-(2-methylpyridin-4-yl)-1H-indole;
5-(5-cyclopropylpyridin-3-yl)-2-(2-methylpyridin-4-yl)-1H-indole;
7-(2-(2-methylpyridin-4-yl)-1H-indol-5-yl)-3,4-dihydro-2H-pyrido[3,2-b][1,4]oxazine;
3-(2-(2-methylpyridin-4-yl)-1H-indol-5-yl)quinoline;
N-methyl-4-(2-(2-methylpyridin-4-yl)-1H-indol-5-yl)pyridin-2-amine;
4-(4-(2-(2-methylpyridin-4-yl)-1H-indol-5-yl)pyridin-2-yl)morpholine;
5-(6-chloro-2-(6-morpholinopyridin-3-yl)-1H-indol-5-yl) quinolin-2-ol;
4-(5-(6-chloro-5-(thieno[2,3-c]pyridin-4-yl)-1H-indol-2-yl)pyridin-2-yl)morpholine;
4-(5-(6-chloro-5-(6-fluoroquinolin-4-yl)-1H-indol-2-yl)pyridin-2-yl)morpholine;
4-(5-(6-chloro-5-(2-methoxyquinolin-5-yl)-1H-indol-2-yl)pyridin-2-yl)morpholine;
4-(5-(6-fluoroquinolin-4-yl)-1H-indol-2-yl)-N,N-dimethylpyridin-2-amine;
4-(5-(isoquinolin-4-yl)-1H-indol-2-yl)-N,N-dimethylpyridin-2-amine;
N,N-dimethyl-4-(5-(quinolin-5-yl)-1H-indol-2-yl)pyridin-2-amine;
3-chloro-2,5-di(pyridin-4-yl)-1H-indole;
2-(2-methylpyridin-4-yl)-5-(pyridin-4-yl)-1H-indole;
5-(5-methoxypyridin-3-yl)-2-(2-methylpyridin-4-yl)-1H-indole;
5-(5-methoxypyridin-3-yl)-2-(pyridin-4-yl)-1H-indole;
5-(5-methoxypyridin-3-yl)-2-(pyridin-3-yl)-1H-indole;
4-(2-(pyridin-4-yl)-1H-indol-5-yl)quinoline;
4-(2-(pyridin-3-yl)-1H-indol-5-yl)quinoline;
2,5-di(pyridin-4-yl)-1H-indole;
2-(pyridin-3-yl)-5-(pyridin-4-yl)-1H-indole;
2,5-di(pyridin-3-yl)-1H-indole;
2-(1-methyl-1H-pyrazol-5-yl)-5-(pyridin-4-yl)-1H-indole;
5-(2-(4-methylpiperazin-1-yl)pyridin-4-yl)-2-(pyridin-4-yl)-1H-indole;
5-(5-methoxypyridin-3-yl)-2-(pyridin-2-yl)-1H-indole;
5-(5-methoxypyridin-3-yl)-2-(pyrimidin-4-yl)-1H-indole;
2-(3-fluoropyridin-4-yl)-5-(5-methoxypyridin-3-yl)-1H-indole;
1-isopropyl-5-(5-methoxypyridin-3-yl)-2-(2-methylpyridin-4-yl)-1H-indole;
4-(5-(6-chloro-5-(5-methoxypyridin-3-yl)-1-methyl-1H-indol-2-yl)pyridin-2-yl) morpholine;
1-(2-methoxyethyl)-2,5-di(pyridin-4-yl)-1H-indole;
1-(2-methoxyethyl)-5-(5-methoxypyridin-3-yl)-2-(2-methylpyridin-4-yl)-1H-indole;
1-methyl-5-(5-methoxypyridin-3-yl)-2-(2-methylpyridin-4-yl)-1H-indole;
1-ethyl-5-(5-methoxypyridin-3-yl)-2-(2-methylpyridin-4-yl)-1H-indole;
1-(cyclopropylmethyl)-5-(5-methoxypyridin-3-yl)-2-(2-methylpyridin-4-yl)-1H-indole;
1-(benzyl)-5-(5-methoxypyridin-3-yl)-2-(2-methylpyridin-4-yl)-1H-indole;
7-(4-chloro-5-(5-methoxypyridin-3-yl)-l-methyl-1H-indol-2-yl)-4-methyl-3,4-dihydro-2H-pyrido[3,2-b][1,4]oxazine;
7-(6-chloro-5-(5-methoxypyridin-3-yl)-l-methyl-1H-indol-2-yl)-4-methyl-3,4-dihydro-2H-pyrido[3,2-b][1,4]oxazine;
1-cyclopropyl-5-(5-methoxypyridin-3-yl)-2-(2-methylpyridin-4-yl)-1H-indole;
1-methyl-2,5-di(pyridin-4-yl)-1H-indole; and
5-(5-methoxypyridin-3-yl)-2-(3-methylpyridin-4-yl)-1H-indole.

A particularly preferred NOX inhibitor for use in the treatment of Alport syndrome is 2-(2-chlorophenyl)-4-[3-(dimethylamino)phenyl]-5-methyl-1H-pyrazolo[4,3-c]pyridine-3,6(2H,5H)-dione, which is also commonly known as, and referred to herein as, setanaxib.

The structure of setanaxib (in non-salt form) is represented below.

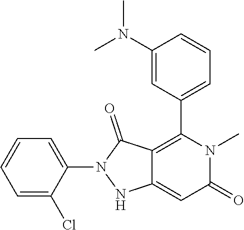 [SMILES: CN(C)c1cccc(-c2c3c(=O)n(-c4ccccc4Cl)[nH]c3cc(=O)n2C)c1]

### Pharmaceutical Compositions and Dosages

The skilled person will understand that, when employed in the uses and methods described herein, compounds of the invention, either used alone or in combination with an ACE inhibitor and/or ARB, may be administered in a manner allowing for systemic absorption, which absorption may occur via a number of possible routes; for example, compounds of the invention may be administered orally, intravenously or intraarterially, intramuscularly, cutaneously, subcutaneously, transmucosally (e.g. sublingually or buccally), rectally, transdermally, nasally, pulmonarily (e.g. by inhalation, tracheally or bronchially), or by any other parenteral route, in the form of a pharmaceutical preparation comprising the compound in a pharmaceutically acceptable dosage form. In particular, compounds of the invention may be administered orally, rectally or intravenously (e.g. by intravenous infusion).

The compounds of the invention will generally be administered in the form of one or more pharmaceutical formulations in admixture with a pharmaceutically acceptable excipient, which may be selected with due regard to the intended route of administration and standard pharmaceutical practice. Such pharmaceutically acceptable excipients may be chemically inert to the active compounds and may have no detrimental side effects or toxicity under the conditions of use. Such pharmaceutically acceptable excipients may also impart an immediate (e.g. rapid), or a modified (e.g. delayed), release of the compounds of the invention.

Suitable pharmaceutical formulations may be commercially available or otherwise are described in the literature (see, for example, Lachman et al, ‘The Theory and Practice of Industrial Pharmacy’, CBS, 4^{th} edition (2015); ‘Remington: The Science and Practice of Pharmacy’, Troy (ed.), Elsevier, 23^{rd} edition (2020); and/or ‘Aulton's Pharmaceutics: The Design and Manufacture of Medicines’, Taylor and Aulton (eds.), Elsevier, 5^{th} edition, 2017), the relevant disclosures in all of which documents are hereby incorporated by reference. Otherwise, the preparation of suitable formulations may be achieved non-inventively by the skilled person using routine techniques.

As described herein, pharmaceutical formulations may be prepared in a manner suitable for the desire route of administration, using techniques and materials known to those skilled in the art. In particular, pharmaceutical formulations may take the form of oral formulations or intravenous formulations (or formulations, e.g. concentrated formulations, suitable for use in the preparation of intravenous formulations).

For example, when intended for oral administration, pharmaceutical formulations comprising compounds of the invention may be provided in the form of a tablet, or an oral powder or solution, each optionally comprising suitable excipients, which may be prepared using techniques known to those skilled in the art. Similarly, when intended for intravenous (I.V.) administration, pharmaceutical formulations comprising compounds of the invention may be provided in the form of solutions suitable for I.V. administration, or as solutions suitable for the preparation of solutions suitable for I.V. administration, which may be prepared using techniques known to those skilled in the art. Similarly, when intended for rectal administration, pharmaceutical formulations comprising compounds of the invention may be provided in the form of a tablet (e.g. a suppository), or a powder or solution, each optionally comprising suitable excipients, which may be prepared using techniques known to those skilled in the art.

Depending on the patient to be treated, the route of administration and the severity of the condition (e.g. the level of pain and the severity of the effects thereof), compounds of the invention may be administered at varying therapeutically effective doses (to the relevant patient in need thereof). Suitable doses may be determined by the skilled person using routine techniques, such as by routine dose titration studies and the like.

Similarly, the amount of the compounds of the invention included in the relevant pharmaceutical formulations may be determined based on the desired dosage of the compound of the invention, the ease of formulation and the route of administration (which may in turn determine the availability of the compound of the invention for systemic adsorption).

Suitable doses of the compounds of the invention may include dosages (e.g. for I.V. administration) in the range of from about 0.05 to 300 mg/kg, such as from about 0.5 to about 200 mg/kg (e.g. about 1 to about 100 mg/kg, such as about 30 mg/kg or about 90 mg/kg). In particular, such doses may administered by I.V. administration over a period of time, such as about one hour. Moreover, such doses may be repeated as necessary, such as in the form of periodic, sequential infusions, which infusions may be of decreasing dose.

For the avoidance of doubt, wherever the word “about” is employed herein, for example in the context of amounts (e.g. doses of active ingredients), it will be appreciated that such variables are approximate and as such may vary by ±10%, for example ±5% and preferably ±2% (e.g. ±1%) about the numbers specified herein. For example, “10±10%” means from 9 to 11.

For the avoidance of doubt, the skilled person will understand that the dose of compounds of the invention administered (e.g. to a human) should be sufficient to effect the desired therapeutic response or effect within (and over) a reasonable timeframe. For example, compounds of the invention may be provided in a form suitable for rapid (i.e. quick or immediate) release of the active ingredient(s), such as in the form of a rapidly disintegrating tablet, which tablets may be formulated using techniques and materials known to those skilled in the art.

In any event, the skilled person will be able to determine routinely the actual dosage which will be most suitable for an individual patient. While the above-mentioned dosages are exemplary of the average case, there can of course be individual instances where higher or lower dosage ranges are merited, and such are within the scope of the invention.

Administration of the compounds of the invention may be continuous (e.g. by continuous I.V. infusion) or intermittent (e.g. by bolus injection or through periodic administration of a tablet or solution), or may be provided in the form of a single dose (e.g. by injection or through administration of a tablet or solution). The dosage form may also be determined by the timing and frequency of administration, and vice versa.

### Combination Product and Associated Kit-of-Parts

As outlined above, in the uses and methods described herein, the compounds of the invention may also be combined with an ACE inhibitor and/or ARB for use in the treatment of Alport syndrome.

Therefore, according to a further aspect of the invention, there is provided a combination product comprising:
(a) a NOX inhibitor, or a pharmaceutically-acceptable salt thereof; and
(b) an ACE inhibitor, or a pharmaceutically-acceptable salt thereof, and/or an ARB, or a pharmaceutically-acceptable salt thereof.
   which combination products are referred to hereinafter as “the combination products according to the invention”.

The NOX inhibitor, the ACE inhibitor and the ARB may be any NOX inhibitor, ACE inhibitor and ARB as disclosed herein.

For example, particular ACE inhibitors include ramipril, benazepril, captopril, enalapril, fosinopril, lisinopril, moexipril, perindopril, quinapril, trandolapril, and combinations thereof. Preferably the ACE inhibitor for use in combination with NOX inhibitors is ramipril.

Particular ARBs include azilsartan, candesartan, eprosartan, sparsentan, irbesartan, losartan, Olmesartan, telmisartan, valsartan, and combinations thereof.

Preferably the combination product comprises:
(a) a NOX inhibitor, or a pharmaceutically-acceptable salt thereof; and
(b) an ACE inhibitor, or a pharmaceutically-acceptable salt thereof.

Even more preferably, the combination product comprises:
(a) setanaxib, or a pharmaceutically-acceptable salt thereof; and
(b) ramipril, or a pharmaceutically-acceptable salt thereof.

For the avoidance of doubt, the ACE inhibitor ramipril may also be referred to by its IUPAC name (2S,3aS,6aS)-1-[(2S)-2-[[(2S)-1-ethoxy-1-oxo-4-phenylbutan-2-yl]amino]propanoyl]-3,3a, 4,5,6,6a-hexahydro-2H-cyclopenta[b]pyrrole-2-carboxylic acid. The structure of the compound (in non-salt form) is represented below.

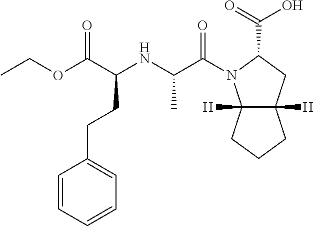 [SMILES: CCOC(=O)[C@H](CCc1ccccc1)N[C@@H](C)C(=O)N1[C@H](C(=O)O)C[C@@H]2CCC[C@@H]21]

Combination products according to the invention provide for the administration of a NOX inhibitor as hereinbefore defined in conjunction with an ACE inhibitor and/or an ARB as hereinbefore defined, and may thus be presented either as separate formulations, wherein at least one of those formulations comprises a NOX inhibitor, and at least one comprises an ACE inhibitor and/or an ARB, or may be presented (i.e. formulated) as a combined preparation (i.e. presented as a single formulation including a NOX inhibitor and an ACE inhibitor or an ARB).

Indeed, the combination product may be presented as three separate formulations, wherein at least one of those formulations comprises a NOX inhibitor, another comprises an ACE inhibitor and another comprises an ARB.

Thus, there is further provided a combination product according to the invention which comprises a kit-of-parts comprising components:
(A) a pharmaceutical formulation including a NOX inhibitor as hereinbefore defined, or a pharmaceutically-acceptable salt thereof, in admixture with a pharmaceutically-acceptable adjuvant, diluent or carrier; and
(B) a pharmaceutical formulation included an ACE inhibitor as hereinbefore defined, or a pharmaceutically-acceptable salt thereof, and/or an ARB, or a pharmaceutically-acceptable salt thereof in admixture with a pharmaceutically-acceptable adjuvant, diluent or carrier,
   which components (A) and (B) are each provided in a form that is suitable for administration in conjunction with the other.

According to a further aspect of the invention, there is provided a method of making a kit of parts as defined above, which method comprises bringing component (A), as defined above, into association with a component (B), as defined above, thus rendering the two components suitable for administration in conjunction with each other.

By bringing the two components “into association with” each other, we include that components (A) and (B) of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Thus, there is further provided a kit of parts comprising:
(I) one of components (A) and (B) as defined herein; together with
(II) instructions to use that component in conjunction with the other of the two components.

The kits of parts described herein may comprise more than one formulation including an appropriate quantity/dose of the NOX inhibitor/salt/solvate, and/or more than one formulation including an appropriate quantity/dose of the ACE inhibitor/salt/solvate, and/or ARB/salt/solvate in order to provide for repeat dosing. If more than one formulation (comprising either active compound) is present, such formulations may be the same, or may be different in terms of the dose of either compound, chemical composition(s) and/or physical form(s).

The combination products according to the invention find utility in the treatment of Alport syndrome.

According to a further aspect of the invention there is provided a method of treatment of Alport syndrome, which method comprises the administration of a combination product according to the invention to a patient in need of such treatment.

With respect to the kits of parts as described herein, by “administration in conjunction with”, we include that respective formulations comprising a NOX inhibitor (or salt/solvate thereof) and an ACE inhibitor (or salt/solvate thereof), and/or ARB (or salt/solvate thereof) are administered, sequentially, separately and/or simultaneously, over the course of treatment of the relevant condition.

Thus, in respect of the combination product according to the invention, the term “administration in conjunction with” includes that the two components of the combination product (the NOX inhibitor and the ACE inhibitor and/or ARB) are administered (optionally repeatedly), either together, or sufficiently closely in time, to enable a beneficial effect for the patient, that is greater, over the course of the treatment of the relevant condition, than if either a formulation comprising the NOX inhibitor, or a formulation comprising the ACE inhibitor and/or ARB, are administered (optionally repeatedly) alone, in the absence of the other component, over the same course of treatment. Determination of whether a combination provides a greater beneficial effect in respect of, and over the course of treatment of, a particular condition will depend upon the condition to be treated or prevented, but may be achieved routinely by the skilled person.

Further, in the context of a kit of parts according to the invention, the term “in conjunction with” includes that one or other of the two formulations may be administered (optionally repeatedly) prior to, after, and/or at the same time as, administration of the other component. When used in this context, the terms “administered simultaneously” and “administered at the same time as” include that individual doses of the NOX inhibitor and the ACE inhibitor and/or ARB are administered within 48 hours (e.g. 24 hours) of each other.

### Preparation of Compounds and Formulations

Compounds of the invention may be obtained commercially or may be prepared using techniques known to those skilled in the art, such as those described in the disclosures referenced herein. For example, setanaxib may be prepared in accordance with the procedure described in Example 62 (scheme 2) of WO 2010/035221, the contents of which (in particular, the procedures as described in the examples provided therein) are hereby incorported by reference.

As described herein, pharmacetical formulations (including those containing more than one active ingredient as described in the third aspect of the invention) may be prepared using techniques known to those skilled in the art. Similarly, a kit-of-parts (as described herein) may be prepared using techniques known to those skilled in the art.

According to further aspects of the invention, there is provided:
a method of preparing a pharmaceutical formulation as described hereinbefore which comprises bringing into admixture (i.e. into the same formulation) components (a) and (b) as described hereinbefore in relation to the relevant aspects of the invention, optionally together with one or more pharmaceutically acceptable excipients; and
a method of preparing a kit-of-parts as described hereinbefore which comprises bringing into association components (A) and (B) as described hereinbefore in relation to the relevant aspects of the invention.

For the avoidance of doubt, by bringing the components “into association with” each other, we include that components (A) and (B) of the kit-of-parts as described hereinbefore may be:
provided as separate formulations (i.e. independently of each other), which are subsequently brought together for use in conjunction with each other in combination therapy; or
packaged and presented as separate components of a “combination pack” for use in conjunction with each other in combination therapy.

When employed in the uses and methods described herein, compounds of the invention may have the advantage that, in the treatment of Alport syndrome, may be more convenient for the physician and/or patient than, be more efficacious than, be less toxic than, have a broader range of activity than, be more potent than, produce fewer side effects than, or that it may have other useful pharmacological properties over, similar treatments (or preventative measures) known in the prior art.

### EXAMPLES

The invention is further illustrated, but not limited, by the following examples and accompanying figures, in which:

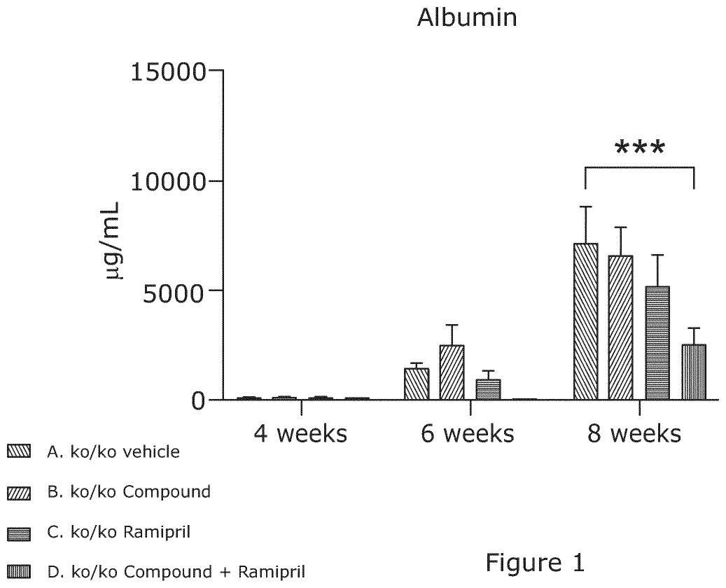

FIG. 1 illustrates the results of a first experiment of analysing albumin content in the urine of homozygous Col4a3 ko/ko mice treated with setanaxib alone (group B; denoted by the term ‘Compound’ in the figure), Ramipril alone (group C), and a combination of setanaxib and Ramipril (group D) as compared to the control (group A; denoted by the term ‘vehicle’ in the figure);

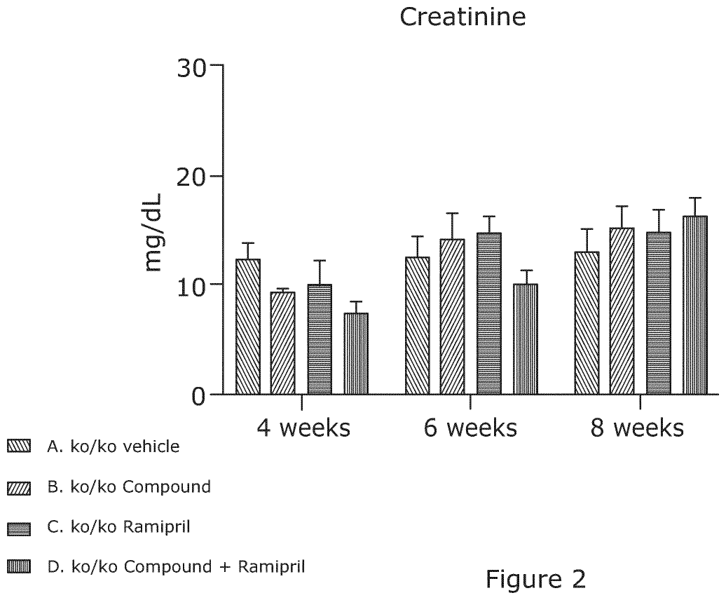

FIG. 2 illustrates the results of a first experiment of analysing creatinine content in the urine of homozygous Col4a3 ko/ko mice treated with setanaxib alone (group B; denoted by the term ‘Compound’ in the figure), Ramipril alone (group C), and a combination of setanaxib and Ramipril (group D) as compared to the control (group A; denoted by the term ‘vehicle’ in the figure);

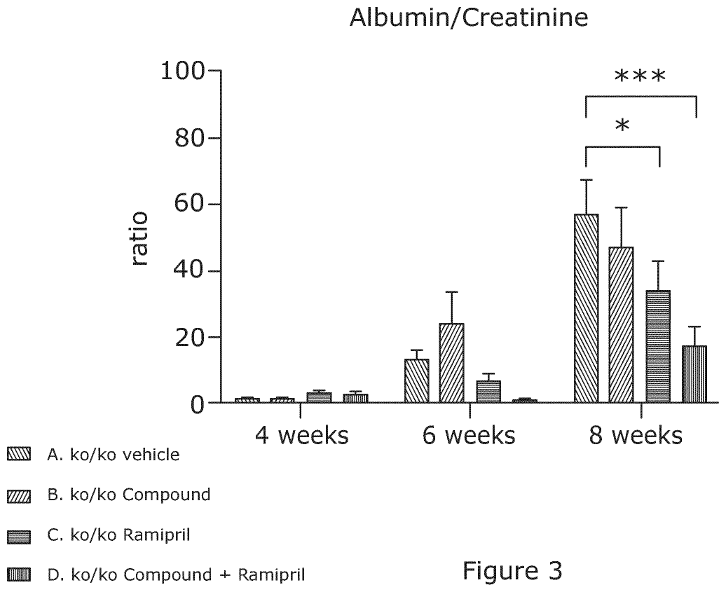

FIG. 3 illustrates the results of a first experiment of analysing albumin/creatinine ratio in the urine of homozygous Col4a3 ko/ko mice treated with setanaxib alone (group B; denoted by the term ‘Compound’ in the figure), Ramipril alone (group C), and a combination of setanaxib and Ramipril (group D) as compared to the control (group A; denoted by the term ‘vehicle’ in the figure);

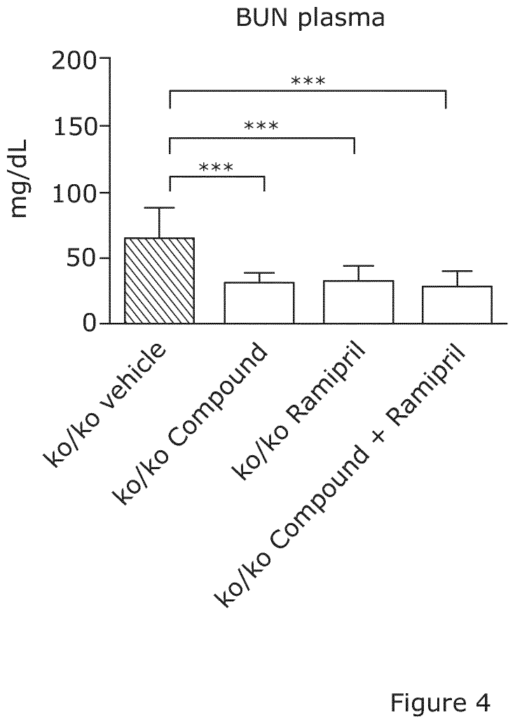

FIG. 4 illustrates the results of a first experiment of analysing Blood Urea Nitrogen (BUN) ratio in the plasma sample of homozygous Col4a3 ko/ko mice treated with setanaxib alone (group B; denoted by the term ‘Compound’ in the figure), Ramipril alone (group C), and a combination of setanaxib and Ramipril (group D) as compared to the control (group A; denoted by the term ‘vehicle’ in the figure);

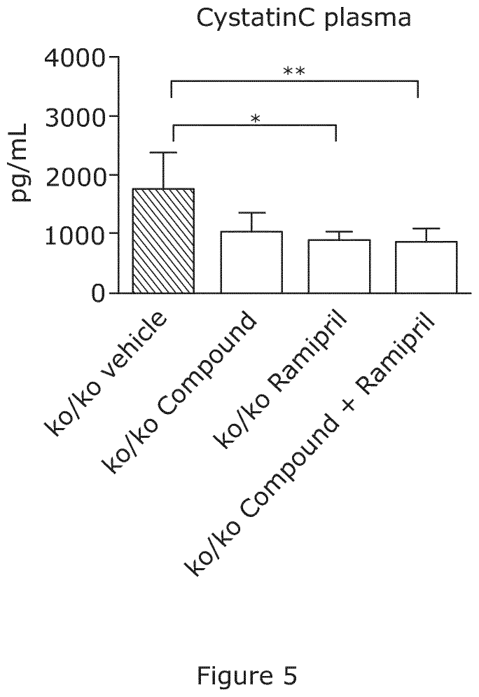

FIG. 5 illustrates the results of analysing Cystatin C in the plasma sample of homozygous Col4a3 ko/ko mice treated with setanaxib alone (group B; denoted by the term ‘Compound’ in the figure), Ramipril alone (group C), and a combination of setanaxib and Ramipril (group D) as compared to the control (group A; denoted by the term ‘vehicle’ in the figure);

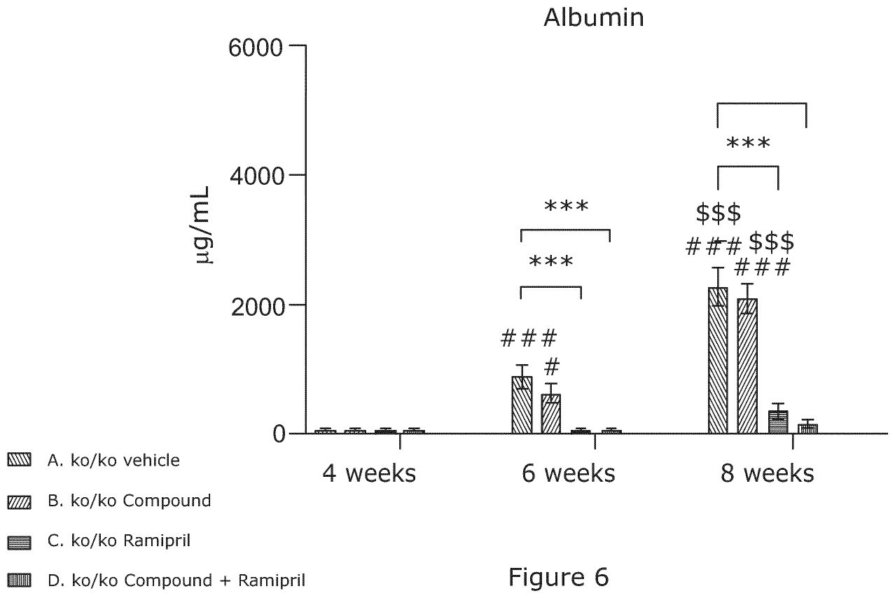

FIG. 6 illustrates the results of a second experiment of analysing albumin content in the urine of homozygous Col4a3 ko/ko mice treated with setanaxib alone (group B; denoted by the term ‘Compound’ in the figure), Ramipril alone (group C), and a combination of setanaxib and Ramipril (group D) as compared to the control (group A; denoted by the term ‘vehicle’ in the figure);

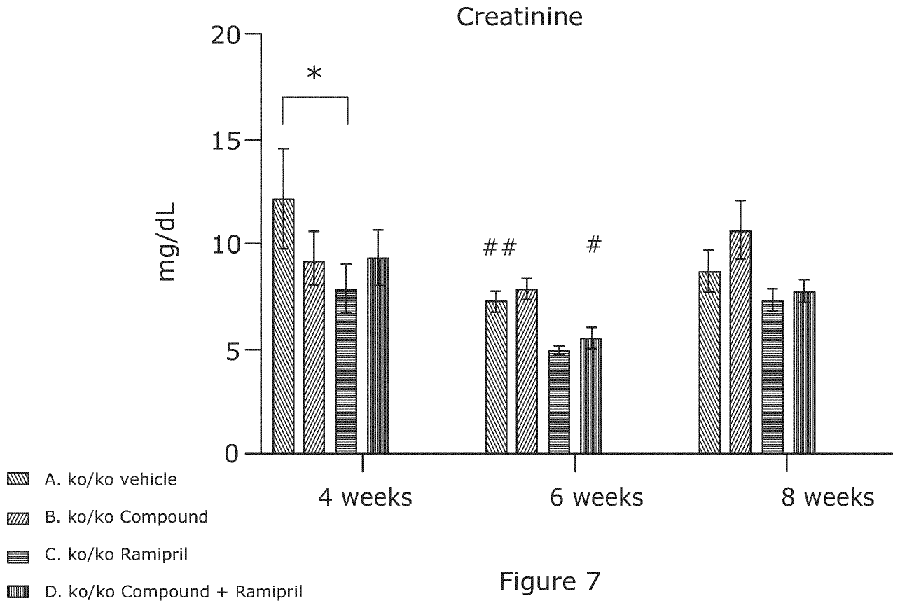

FIG. 7 illustrates the results of a second experiment of analysing creatinine content in the urine of homozygous Col4a3 ko/ko mice treated with setanaxib alone (group B; denoted by the term ‘Compound’ in the figure), Ramipril alone (group C), and a combination of setanaxib and Ramipril (group D) as compared to the control (group A; denoted by the term ‘vehicle’ in the figure);

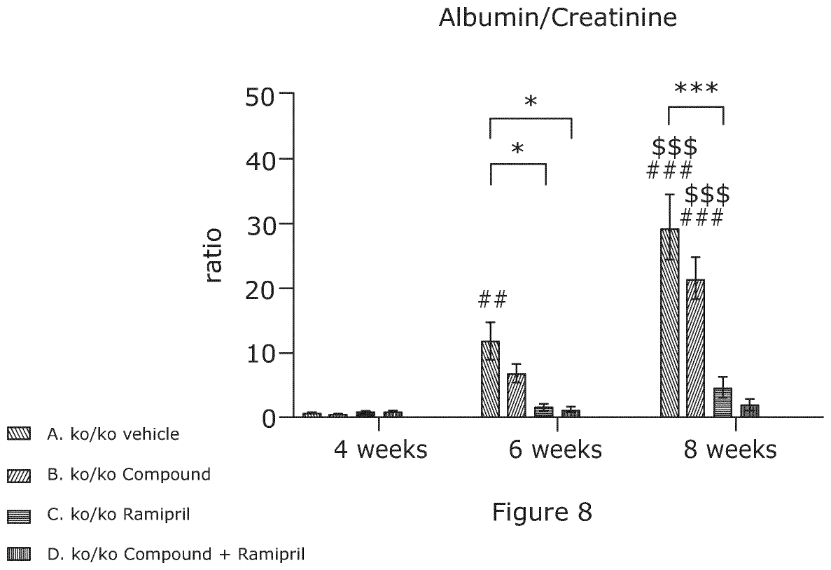

FIG. 8 illustrates the results of a second experiment of analysing albumin/creatinine ratio in the urine of homozygous Col4a3 ko/ko mice treated with setanaxib alone (group B; denoted by the term ‘Compound’ in the figure), Ramipril alone (group C), and a combination of setanaxib and Ramipril (group D) as compared to the control (group A; denoted by the term ‘vehicle’ in the figure); and

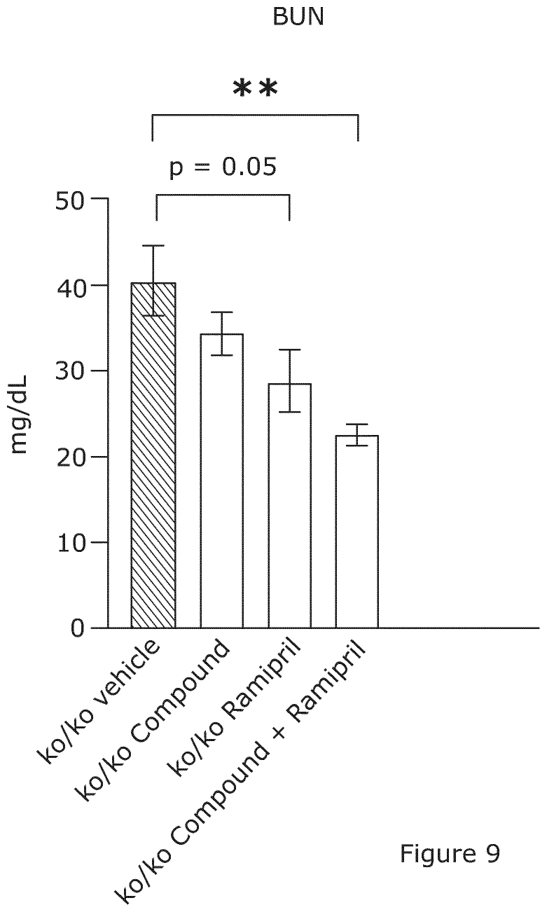

FIG. 9 illustrates the results of a second experiment of analysing Blood Urea Nitrogen (BUN) ratio in the plasma sample of homozygous Col4a3 ko/ko mice treated with setanaxib alone (group B; denoted by the term ‘Compound’ in the figure), Ramipril alone (group C), and a combination of setanaxib and Ramipril (group D) as compared to the control (group A; denoted by the term ‘vehicle’ in the figure).

### EXAMPLE 1

### Study in a Col4A3 Alport Mouse Model

### SUMMARY

The aim of the study was to examine the effects of setanaxib, a NOX (e.g., NOX1/4) inhibitor in a mouse model of Alport syndrome in Col4a3 ko/ko mice.

The study was duplicated and each study is referred to herein as the first experiment and the second experiment.

Mice homozygous for the Col4a3 ko/ko targeted mutation are a model for autosomal-recessive Alport syndrome (Cosgrove et al. Kidney Int. 2007, 71(7), p. 615-618). Animals bred on a 129/SvJ background develop glomerulonephritis and die at about 8.5 weeks of age (Jackson Laboratory strain: #002908). Gross et al. Kidney Int. 2003, 63(2), p. 438-446) reported a mean survival of 10 weeks. Since homozygous Col4a3 ko/ko mice reflect key features of Alport syndrome, this mouse model is useful to test potential drug candidates.

A number of 40 homozygous Col4a3 ko/ko mice, allocated to 4 groups (vehicle, setanaxib, Ramipril, and setanaxib+Ramipril combination) with n=10 animals each, were used for the study. Starting at an age of 4 weeks, animals were treated with vehicle and/or compound daily per oral gavage for 4 weeks. The reference compound Ramipril was administered via drinking water, prepared on a weekly basis, but changed at the latest after 4 days. Additionally, one wild-type control group (Col4A3+/+) without any treatment was enclosed in the study with n=6 animals.

Clinical signs and termination criteria were monitored on a daily basis required upon the age of 8 weeks until tissue collection.

Urine samples were collected at 3 points of time (baseline (at an age of 4 weeks), at 6 and 8 weeks of age after respectively 2 and 4 weeks of treatment)). Albumin and Creatinine levels were evaluated by ELISA in urine samples of 8 animals per treatment group.

At study end the animals were euthanized by i.p. injection of pentobarbital and terminal blood was collected by heart puncture in EDTA coated tubes. Blood plasma was collected by centrifugation (3000×g for 10 minutes at room temperature) and 50 μL plasma aliquots were transferred to 1.5 mL tubes, frozen on dry ice and stored at −80° C.

Treatment 40 homozygous Col4a3 ko/ko mice, allocated to 4 groups with n=10 animals each, were used for this study. Animals were treated with vehicle or compound daily per oral gavage for 4 weeks starting at an age of 4 weeks. The reference compound Ramipril was administered via drinking water, prepared on a weekly basis, but changed latest after 4 days. Additionally, one wild-type control group without any treatment was enclosed in the study with n=6 animals. The treatment groups are outlined in the table below:

| | | | | | | | Route |
|---|---|---|---|---|---|---|---|
| | | | | | | Treatment | of |
| Group | N | Genotype | Treatment | Dosage | Frequency | duration | application |
| A | 10 | ko/ko | Vehicle | — | daily | 4 weeks | oral via |
| | | | | | | | gavage |
| B | 10 | ko/ko | Compound | 60 mg/kg b.w. | daily | 4 weeks | oral via |
| | | | | | | | gavage |
| C | 10 | ko/ko | Ramipril | 10 mg/kg b.w. | ad libitum | 4 weeks | oral via |
| | | | | | | | drinking |
| | | | | | | | water |
| D | 10 | ko/ko | Compound + | 60 mg/kg b.w. | daily | 4 weeks | Compound: |
| | | | Ramipril | 10 mg/kg b.w. | ad libitum | | oral via |
| | | | | | | | gavage |
| | | | | | | | Ramipril: |
| | | | | | | | oral |
| | | | | | | | via |
| | | | | | | | drinking |
| | | | | | | | water |
| E | 6 | wt | untreated | N/A | N/A | N/A | N/A |

### Urine Sampling

Urine samples were collected from all animals of groups A to D by direct withdrawal of the produced urine using a pipette at baseline prior to treatment start and at an age of 6 and 8 weeks, respectively. In case no urine was produced during the fixation process, massage of the abdomen was allowed. Urine was collected in Eppendorf Tubes, spun at 800×g at room temperature for 10 minutes and supernatant was transferred into a fresh tube and frozen at −20° C. until use.

### Biochemistry

Creatinine and Albumin levels in urine supernatants, collected from 8 animals each group A to D at 3 timepoints, at baseline, 6 and 8 weeks of age (n=96), were analyzed.

### Creatinine Level Determination

Creatinine levels in urine samples of all animals (n=8 per group, 4 groups, 3 time points, total n=96) were analyzed. For that purpose, an enzymatic assay from Diazyme® (DZ072B) was used. The analysis was performed according to manufacturer protocol with minor modifications introduced for use in 96 well plates and readout in standard plate photometer (Cytation 5, Biotek).

### Albumin Level Determination

Albumin levels in urine supernatants, collected from each animal per group at 3 time points (total n=96) were analyzed using an Albumin Mouse ELISA Kit (ab108792; Abcam) according to manufacturer protocol.

Briefly, urine samples were centrifuged at 800×g for 10 minutes and diluted into Diluent N. 50 μL of albumin standard or sample was added per well and incubated for 2 hours. After a washing step, 50 μL of biotinylated albumin antibody was added to each well and incubated for 1 hour. The microplate was washed, followed by the addition of 50 μL of streptavidin-peroxidase conjugate per well and incubation for 30 minutes. Thereafter, the microplate was washed again and incubated with 50 μL of chromogen substrate per well for 15 minutes. The reaction was stopped by adding 50 μL stop solution per well. Albumin levels were determined by reading the absorbance at 450 nm immediately after adding the stop solution.

### Plasma Sampling

After confirmation of deep anaesthesia, the thorax was opened and blood was collected by heart puncture with a 23-gauge needle. The needle was removed and the blood was transferred to the sample tube (MiniCollect® K2EDTA (potassium ethylenediaminetetraacetic acid)). The tube had to be inverted thoroughly to facilitate homogeneous distribution of the EDTA and prevent clotting. The blood samples were centrifuged at 3000×g for 10 minutes at room temperature (22° C.). Plasma was transferred to a pre-labelled 1.5 ml LoBind Eppendorf tube (50 μl aliquots+rest), frozen on dry ice and stored at −80° C.

### Blood Urea Nitrogen (BUN) Colorimetric Detection

BUN plasma levels of all animals at the end of the study were analyzed. For that purpose, an assay from Invitrogen™ (catalogue number: EIABUN) was used. The analysis was performed according to manufacturer protocol.

### Quantitative Detection of Mouse Cystatin C

Cystatin C plasma levels of all animals at the end of the study were analyzed. For that purpose, an ELISA kit from Abcam® (catalogue number: ab119590) was used. The analysis was performed according to manufacturer protocol.

### Results

### Albumin Levels

Evaluation of Albumin levels in the urine of homozygous Col4a3 ko/ko mice showed an increase over time in both the first and second experiment, and the results can be seen in FIGS. 1 and 6. At an age of 4 weeks (baseline) similar Albumin values within all groups were observed. While setanaxib treatment (group B) revealed a trend towards increased Albumin levels compared to vehicle treated animals at an age of 6 weeks, Ramipril (group C) slightly reduced the amount of albumin in the urine in the first experiment and in the second experiment the albumin in urine levels were reduced to a greater extent. Interestingly, in both experiments the combination of Ramipril and setanaxib (group D) decreased Albumin levels compared to vehicle treated mice. At an age of 8 weeks, Albumin levels were significantly decreased after treatment of homozygous Col4a3 ko/ko mice with Ramipril and setanaxib (group D) compared to control mice (group A), and treatment with setanaxib alone showed a decrease in average Albumin levels.

### Creatinine Levels

Analysis of Creatinine levels in the urine revealed no significant differences between the groups at all tested time points in both the first and second experiment. The results may be seen in FIGS. 2 and 7.

### Albumin/Creatinine Ratio

Calculation of Albumin to Creatinine ratio showed a trend in both the first and second experiment towards an increase after treatment with setanaxib in the urine of 6 weeks old Col4a3 ko/ko mice (group B) compared to vehicle treated animals (group A) as detailed in FIGS. 3 and 8. In contrast, Ramipril treatment (group C) reduced the Albumin to Creatinine ratio, but without reaching significance in the first experiment (in the second experiment the reduction did reach significance). Combination treatment (Ramipril+setanaxib) further decreased the ratio, with the first experiment reaching significance after performing statistical analysis with unpaired t-test. At an age of 8 weeks, the Albumin to Creatinine ratio was significantly decreased in the Col4a3 ko/ko mice treated with Ramipril alone (group C) or in combination with the setanaxib (group D) compared to vehicle treated animals, and treatment with setanaxib alone showed an average decrease (group B).

### BUN and Cystatin C Levels in Plasma Sample

The results of the study of Blood Urea Nitrogen (BUN) and Cystatin C in plasma samples are illustrated in FIGS. 4 and 5, respectively for the first experiment. The results of the study of Blood Urea Nitrogen (BUN) in plasma samples for the second experiment are illustrated in FIG. 9.

In the first experiment, treatment with setanaxib alone, ramipril alone and a combination of the two showed a significant decrease in BUN and Cystatin C plasma levels compared to the vehicle alone. The decrease was similar for all treatment groups. In the second experiment, the combination of ramipril and setanaxib showed a significant decrease in BUN.

1. (canceled)

2. The method according to claim 20, wherein the NOX inhibitor is a NOX4 inhibitor, a NOX4/1 inhibitor or a NOX1 inhibitor, or a pharmaceutically acceptable salt thereof.

3. The method according to claim 20, wherein the NOX inhibitor is a pyrazolo pyridine NOX4 or NOX4/1 inhibitor, a pyrazolinedione NOX4 or NOX4/1 inhibitor, an amidothiazole NOX1 or NOX4/1 inhibitor, or an indole NOX4/1 inhibitor, or a pharmaceutically acceptable salt thereof.

4. The method according to claim 20, wherein the NOX inhibitor is a 2,5-disubstituted benzoxazole or a benzothiazole NOX4 inhibitor, or a pharmaceutically acceptable salt thereof.

5. The method according to claim 20, wherein the NOX inhibitor is a NOX inhibitor according to Formula (I): 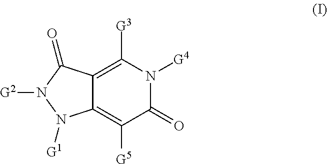
wherein:
G¹ is selected from the group consisting of:
H;
optionally substituted alkyl, such as aminocarbonyl alkyl (e.g. phenylacetamide);
optionally substituted C₃-C₈-cycloalkyl alkyl;
optionally substituted heterocycloalkyl alkyl;
aryl alkyl, such as phenyl alkyl, for example phenyl methyl or 3-methyl phenyl methyl;
substituted aryl alkyl, such as substituted phenyl alkyl, for example substituted phenyl methyl (e.g. 4-fluorobenzyl, 2-chlorobenzyl, 4-chlorobenzyl, 4-methyl benzyl, or 4-bromobenzyl);
heteroaryl alkyl; and
substituted heteroaryl alkyl, such as substituted pyridine alkyl, for example pyridine-2-yl methyl;
G² is selected from the group consisting of:
H;
optionally substituted alkyl;
optionally substituted alkenyl;
optionally substituted alkynyl;
optionally substituted aryl, such as phenyl (e.g. 4-fluorophenyl, 4-methoxyphenyl, 4-nitrophenyl, 2-chlorophenyl, 2-methyl phenyl, 4-(trifluoromethyl)phenyl, 4-(trifluoromethoxy)phenyl, 2,5-difluorophenyl, or 2-methoxyphenyl);
optionally substituted alkyl aryl;
optionally substituted aryl alkyl;
heteroaryl, such as benzothiazolyl, or pyridinyl;
substituted heteroaryl, such as substituted benzothiazolyl (e.g. 1,3-benzothiazol-2-yl), or substituted pyridinyl (e.g. pyridin-2-yl);
optionally substituted alkyl heteroaryl;
optionally substituted heteroaryl alkyl;
optionally substituted alkenyl aryl;
optionally substituted aryl alkenyl;
optionally substituted alkenyl heteroaryl;
optionally substituted heteroaryl alkenyl;
optionally substituted C₃-C₈-cycloalkyl;
optionally substituted heterocycloalkyl;
optionally substituted alkyl C₃-C₈-cycloalkyl;
optionally substituted C₃-C₈-cycloalkyl alky;
optionally substituted alkyl heterocycloalkyl; and
optionally substituted heterocycloalkyl alkyl;
G³ is selected from the group consisting of:
H;
optionally substituted alkyl, such as optionally substituted methyl or optionally substituted ethyl;
optionally substituted alkenyl;
optionally substituted alkynyl;
optionally substituted aryl, such as optionally substituted phenyl;
optionally substituted alkyl aryl;
optionally substituted aryl alkyl;
optionally substituted heteroaryl;
optionally substituted alkyl heteroaryl;
optionally substituted heteroaryl alkyl;
optionally substituted alkenyl aryl;
optionally substituted aryl alkenyl;
optionally substituted alkenyl heteroaryl;
optionally substituted heteroaryl alkenyl;
optionally substituted C₃-C₈-cycloalkyl;
optionally substituted heterocycloalkyl;
optionally substituted alkyl C₃-C₈-cycloalkyl;
optionally substituted C₃-C₈-cycloalkyl alkyl;
optionally substituted alkyl heterocycloalkyl; and
optionally substituted heterocycloalkyl alkyl;
G⁴ is selected from the group consisting of:
H;
optionally substituted alkyl, such as optionally substituted pentyl (e.g., isopentyl);
optionally substituted heteroalkyl;
optionally substituted alkoxy, such as optionally substituted methoxy; (e.g. 2-methoxyethyl);
optionally substituted alkenyl;
optionally substituted alkynyl;
optionally substituted aryl;
optionally substituted alkyl aryl;
aryl alkyl, such as benzyl or phenyl ethyl (e.g., 2-phenyl ethyl);
substituted aryl alkyl such as substituted benzyl (e.g. benzoic acid methyl) or substituted phenyl ethyl (e.g. 4-methoxyphenyl ethyl);
optionally substituted heteroaryl;
optionally substituted alkyl heteroaryl;
optionally substituted heteroaryl alkyl, such as optionally substituted thiophenyl alkyl (such as optionally substituted thiophenyl methyl, for example thiophen-2-yl methyl), optionally substituted imidazolyl alkyl (such as optionally substituted imidazolyl ethyl, for example imidazol-4-yl ethyl), optionally substituted indolyl alkyl (such as optionally substituted indolyl ethyl, for example indol-3-yl ethyl), optionally substituted furanyl alkyl (such as optionally substituted furanyl methyl, for example furan-2-yl methyl), optionally substituted benzodioxolyl alkyl (such as optionally substituted benzodioxolyl methyl, for example 1,3-benzodioxol-5-yl methyl), or optionally substituted pyridinyl methyl (such as pyridine-3-yl methyl or pyridin-2-yl methyl);
optionally substituted alkenyl aryl;
optionally substituted aryl alkenyl;
optionally substituted alkenyl heteroaryl;
optionally substituted heteroaryl alkenyl;
optionally substituted C₃-C₈-cycloalkyl;
heterocycloalkyl, such as morpholinyl (e.g., 5-morpholin-4-yl), piperazinyl (e.g. 4-methyl piperazinyl), or piperidinyl (e.g. 4-methylbenzyl) piperidin-4-yl);
substituted heterocycloalkyl, such as substituted morpholinyl (e.g. 5-morpholin-4-yl), substituted piperazinyl (e.g. 4-methyl piperazinyl), or substituted piperidinyl (e.g. 4-methylbenzyl) piperidin-4-yl);
optionally substituted alkyl C₃-C₈-cycloalkyl;
optionally substituted C₃-C₈-cycloalkyl alkyl;
optionally substituted alkyl heterocycloalkyl;
heterocycloalkyl alkyl, such as morpholinyl alkyl (for example, morpholinyl ethyl, or morpholinyl propyl, in particular 2-morpholin-4-yl-ethyl, or 3-(morpholin-4-yl)propyl), piperazinyl alkyl (such as piperazinyl ethyl, for example 2-(4-acetylpiperazin-1-yl)ethyl, or 2-(4-hexanoyl piperazin-1-yl)ethyl), or pyrrolidinyl alkyl (such as pyrrolidinyl propyl, for example 3-(2-oxopyrrolidin-1-yl)propyl), or tetrahydro furanyl alkyl (such as tetrahydrofuranyl methyl, for example tetrahydrofuran-2-yl methyl); and
substituted heterocycloalkyl alkyl such as substituted morpholinyl alkyl (for example substituted morpholinyl ethyl, or substituted morpholinyl propyl, in particular substituted 2-morpholin-4-yl-ethyl, or substituted 3-(morpholin-4-yl)propyl), substituted piperazinyl alkyl (such as substituted piperazinyl ethyl, for example substituted 2-(4-acetylpiperazin-1-yl) ethyl or substituted 2-(4-hexanoyl piperazin-1-yl)ethyl), or substituted pyrrolidinyl alkyl (such as substituted pyrrolidinyl propyl, for example substituted 3-(2-oxopyrrolidin-1-yl)propyl), or substituted tetrahydro furanyl alkyl (such as substituted tetrahydrofuranyl methyl, for example substituted tetrahydrofuran-2-yl methyl);
G⁵ is selected from the group consisting of:
H;
optionally substituted alkyl;
optionally substituted alkenyl;
optionally substituted alkynyl;
optionally substituted aryl;
optionally substituted alkyl aryl;
optionally substituted aryl alkyl;
optionally substituted heteroaryl;
optionally substituted alkyl heteroaryl;
optionally substituted heteroaryl alkyl;
optionally substituted alkenyl aryl;
optionally substituted aryl alkenyl;
optionally substituted alkenyl heteroaryl;
optionally substituted heteroaryl alkenyl;
optionally substituted C₃-C₈-cycloalkyl;
optionally substituted heterocycloalkyl;
optionally substituted alkyl C₃-C₈-cycloalkyl;
optionally substituted C₃-C₈-cycloalkyl alkyl;
optionally substituted alkyl heterocycloalkyl; and
optionally substituted heterocycloalkyl alkyl;
as well as pharmaceutically acceptable salts thereof.

6. The method according to claim 20, wherein the NOX inhibitor is a NOX inhibitor according to Formula (II): 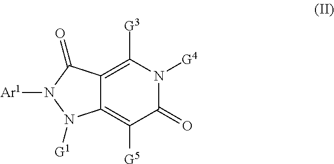
wherein:
Ar¹ is selected from the list consisting of:
phenyl;
phenyl substituted by a halogen, such as chloro (for example wherein Ar¹ is 2-chlorophenyl); and
phenyl substituted by an alkoxy, such as methoxy
G¹ and G⁵ are H;
G³ is selected from the list consisting of:
optionally substituted C₁-C₆ alkyl, such as optionally substituted methyl;
phenyl;
phenyl substituted by halogen, amino, alkyl amino, or alkoxy, for example wherein the phenyl is 3-chlorophenyl, 4-chlorophenyl, 2-fluorophenyl, 3-fluorophenyl, 4-fluorophenyl, 4-chloro-2-fluorophenyl, 5-chloro-2-fluorophenyl, 3-dimethylaminophenyl, 2-tri-methyl amino phenyl, 3-methyl amino phenyl, 3-amino phenyl, or 4-methoxy phenyl;
G⁴ is selected from the list consisting of:
H;
C₁-C₆ alkyl, such as methyl,
substituted C₁-C₆ alkyl, such as substituted methyl, optionally wherein the C₁-C₆ alkyl is substituted by an alkoxy, such as wherein the substituted C₁-C₆ alkyl is methoxy ethyl, for example 2-methoxy ethyl;
heteroaryl C₁-C₆ alkyl, such as pyridinyl C₁-C₆ alkyl (for example pyridinyl methyl, in particular pyridinyl-2-yl methyl, pyridinyl-3-yl methyl, 6-methoxypyridin-3-yl methyl, 2-methoxypyridin-4-yl methyl), or pyrazinyl C₁-C₆ alky, such as pyrazinyl-2-ylmethyl;
substituted heteroaryl C₁-C₆ alkyl, such as substituted pyridinyl C₁-C₆ alkyl (for example substituted pyridinyl methyl, in particular substituted pyridinyl-2-yl methyl, substituted pyridinyl-3-yl methyl, substituted 6-methoxypyridin-3-yl methyl, substituted 2-methoxypyridin-4-yl methyl), or substituted pyrazinyl C₁-C₆ alky, such as substituted pyrazinyl-2-ylmethyl;
alkoxy C₁-C₆ alkyl, such as methoxy ethyl (for example 2-methoxy ethyl); and
substituted C₁-C₆ alkyl, such as substituted methoxy ethyl (for example substituted 2-methoxy ethyl)
or G³ and G⁴ form together an optionally substituted 7-membered heterocycloalkyl ring comprising two nitrogen atoms, and where the two nitrogens are attached through a optionally substituted C₁-C₃ alkyl moiety,
as well as tautomers, geometrical isomers, optically active forms and pharmaceutically acceptable salts thereof.

7. The method according to claim 20, wherein the NOX inhibitor is a NOX inhibitor according to Formula (III): 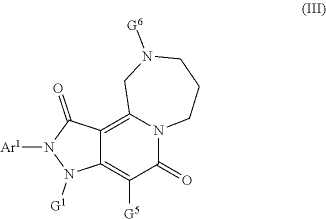
wherein:
Ar¹, G¹ and G⁵ are as defined in claim 5 or claim 6;
G⁶ is selected from the group consisting of:
C₁-C₆ alkyl;
substituted C₁-C₆ alkyl;
aryl C₁-C₆ alkyl, such as benzyl;
substituted aryl C₁-C₆ alkyl, such as substituted phenyl C₁-C₆ alkyl, for example 2-chlorobenzyl, 3-chlorobenzyl, 4-chlorobenzyl, 2-methoxybenzyl, 3-methoxybenzyl, 4-methoxybenzyl);
heteroaryl C₁-C₆ alkyl, such as pyridinyl C₁-C₆ alkyl, for example pyridinyl methyl (such as pyridinyl-2-yl methyl, or pyridinyl-3-yl methyl), or furanyl C₁-C₆ alkyl, for example furanyl methyl (such as furan-3-yl methyl);
substituted heteroaryl C₁-C₆ alkyl, such as substituted pyridinyl C₁-C₆ alkyl, for example substituted pyridinyl methyl (such as substituted pyridinyl-2-yl methyl, or substituted pyridinyl-3-yl methyl), or substituted furanyl C₁-C₆ alkyl, for example substituted furanyl methyl (such as substituted furan-3-yl methyl);
as well as tautomers, geometrical isomers, optically active forms and pharmaceutically acceptable salts thereof.

8. The method according to claim 20, wherein the NOX inhibitor is a NOX inhibitor according to Formula (IV): 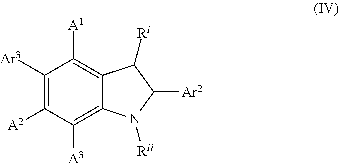
wherein:
A¹, A² and A³ are independently selected from the list consisting of:
hydrogen;
halogen;
CN;
CF₃;
CHF₂;
optionally substituted C₁-C₆ alkyl;
optionally substituted C₃-C₈ cycloalkyl;
optionally substituted heteroalkyl;
optionally substituted heterocycloalkyl;
optionally substituted alkoxy;
optionally substituted amino;
optionally substituted carboxy; and
optionally substituted alkoxycarbonyl; and
Rⁱ is selected from the group consisting of:
hydrogen;
halogen;
CN;
CF₃;
CHF₂;
optionally substituted C₁-C₆ alkyl;
optionally substituted C₃-C₈ cycloalkyl;
optionally substituted heteroalkyl;
optionally substituted heterocycloalkyl; and
optionally substituted carboxy;
Rⁱⁱ is selected from the list consisting of:
hydrogen;
optionally substituted C₁-C₆ alkyl;
C₃-C₈ cycloalkyl;
optionally substituted halo C₁-C₆ alkyl;
optionally substituted halo C₃-C₈ cycloalkyl;
optionally substituted aryl C₁-C₆ alkyl;
optionally substituted heteroaryl C₁-C₆ alkyl;
optionally substituted heteroalkyl;
optionally substituted heterocycloalkyl;
substituted heterocycloalkyl;
optionally substituted heterocycloalkyl C₁-C₆ alkyl;
optionally substituted hydroxy C₁-C₆ alkyl;
optionally substituted alkoxy C₁-C₆ alkyl;
optionally substituted alkoxycarbonyl C₁-C₆ alkyl;
optionally substituted aminocarbonyl C₁-C₆ alkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl; and
optionally substituted halo C₃-C₈ cycloalkyl;
Ar³ is selected from: 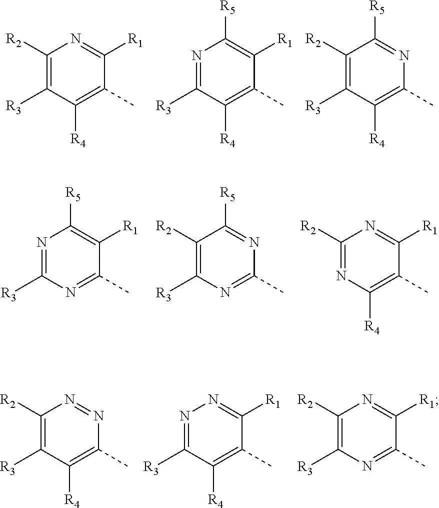
R¹ and R⁴ are independently selected from the list consisting of:
hydrogen;
halogen;
CN;
CF₃;
CHF₂;
NH₂;
optionally substituted C₁-C₆ alkyl;
optionally substituted C₃-C₈ cycloalkyl;
alkoxy;
amino;
optionally substituted heterocycloalkyl;
carboxy;
optionally substituted alkoxy C₁-C₆ alkyl;
optionally substituted alkoxy C₃-C₈ cycloalkyl;
optionally substituted amino C₁-C₆ alkyl;
optionally substituted amino C₃-C₈ cycloalkyl;
optionally substituted amino heterocycloalkyl C₁-C₆ alkyl;
optionally substituted carboxy C₃-C₈ cycloalkyl; and
optionally substituted aminocarbonyl C₁-C₆ alkyl;
R² is selected from the list consisting of:
hydrogen;
halogen;
CF₃;
CHF₂;
NH₂;
optionally substituted C₁-C₆ alkyl;
optionally substituted C₃-C₈ cycloalkyl;
optionally substituted alkoxy;
amino;
optionally substituted heterocycloalkyl;
carboxy;
optionally substituted alkoxy C₁-C₆ alkyl;
optionally substituted alkoxy C₃-C₈ cycloalkyl;
optionally substituted amino C₁-C₆ alkyl;
optionally substituted amino C₃-C₈ cycloalkyl;
optionally substituted amino heterocycloalkyl C₁-C₆ alkyl;
optionally substituted C₃-C₈ carboxy cycloalkyl; and
optionally substituted aminocarbonyl C₁-C₆ alkyl;
R³ is selected from the list consisting of:
hydrogen;
halogen;
CF₃;
CHF₂;
NH₂;
optionally substituted C₁-C₆ alkyl;
optionally substituted C₃-C₈ cycloalkyl;
optionally substituted alkoxy;
amino;
optionally substituted heterocycloalkyl;
carboxy;
optionally substituted alkoxy C₁-C₆ alkyl;
optionally substituted alkoxy C₃-C₈ cycloalkyl;
optionally substituted amino C₁-C₆ alkyl;
optionally substituted amino C₃-C₈ cycloalkyl;
optionally substituted amino heterocycloalkyl C₁-C₆ alkyl;
optionally substituted C₃-C₈ carboxy cycloalkyl; and
optionally substituted aminocarbonyl C₁-C₆ alkyl;
R⁵ is selected from the group consisting of:
hydrogen;
halogen;
CF₃;
CHF₂;
NH₂;
optionally substituted C₁-C₆ alkyl;
optionally substituted C₃-C₈ cycloalkyl;
optionally substituted alkoxy;
amino;
optionally substituted heterocycloalkyl;
carboxy;
optionally substituted alkoxy C₁-C₆ alkyl;
optionally substituted alkoxy C₃-C₈ cycloalkyl;
optionally substituted amino C₁-C₆ alkyl;
optionally substituted amino C₃-C₈ cycloalkyl;
optionally substituted amino heterocycloalkyl C₁-C₆ alkyl;
optionally substituted C₃-C₈ carboxy cycloalkyl; and
optionally substituted aminocarbonyl C₁-C₆ alkyl,
wherein when one from R¹, R², R³ and R⁴ is not H, the other from this group are H or any of R¹, R², R³, R⁴ and R⁵ can be linked together to form an optionally substituted bicyclic heteroaryl;
Ar³ is also selected from an optionally substituted bicyclic heteroaryl, in particular from the group consisting of: 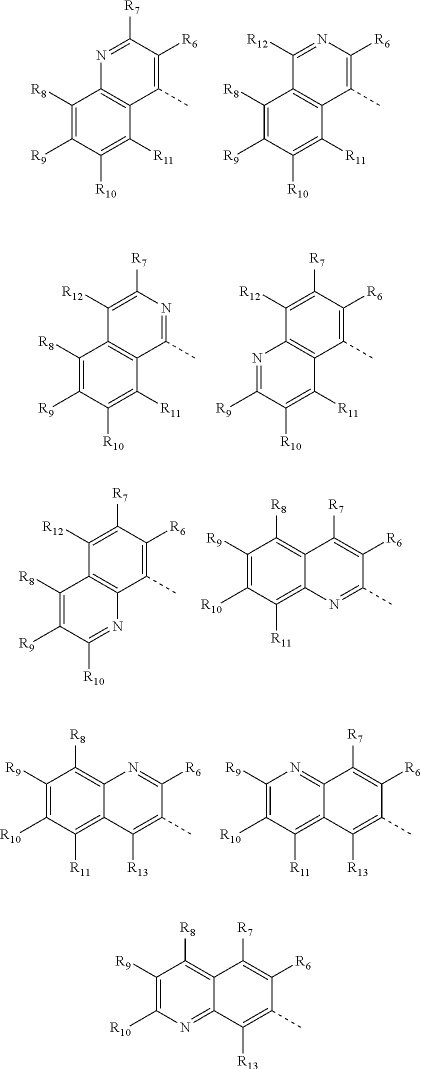
wherein R⁶, R⁷, R⁸, R⁹, R¹⁰, R¹¹, R¹² and R¹³ are each independently selected from the list consisting of:
hydrogen;
halogen;
hydroxy;
CN;
CF₃,
CHF₂;
NH₂;
alkoxy;
amino;
carboxy;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
optionally substituted alkoxy C₁-C₆ alkyl;
optionally substituted amino C₁-C₆ alkyl;
optionally substituted carboxy C₁-C₆ alkyl;
optionally substituted aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl;
optionally substituted aminocarbonyl C₃-C₈ cycloalkyl;
acylamino;
ureido;
sulfonyl; and
sulfonylamino;
R¹⁴ is selected from the list consisting of:
hydrogen;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
optionally substituted alkoxy C₁-C₆ alkyl;
optionally substituted amino C₁-C₆ alkyl,
carboxy C₁-C₆ alkyl;
optionally substituted aminocarbonyl C₁-C₆ alkyl;
optionally substituted heterocycloalkyl C₃-C₈ cycloalkyl;
aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl, and
optionally substituted aminocarbonyl C₃-C₈ cycloalkyl;
Ar² is selected from the following group: 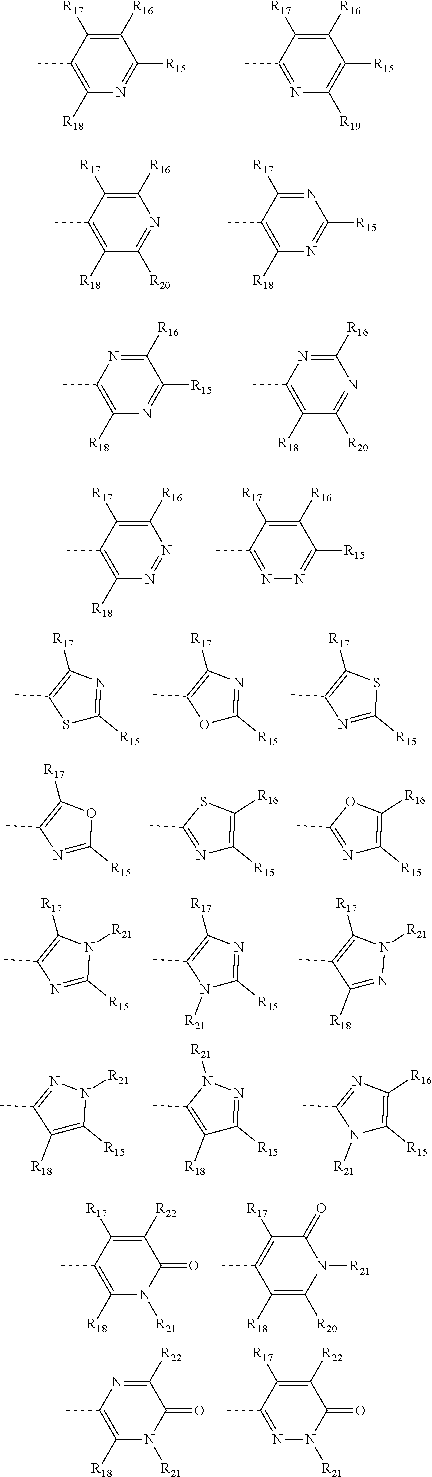 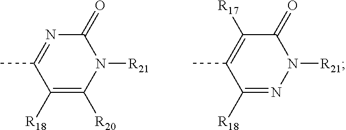
wherein R¹⁵, R¹⁶ and R¹⁹ are independently selected from the list consisting of:
hydrogen,
halogen;
hydroxy,
CN;
CF₃;
CHF₂;
NH₂;
alkoxy;
amino;
carboxy;
aminocarbonyl;
alkoxy carbonyl;
optionally substituted C₁-C₆ alkyl;
optionally substituted C₃-C₈ cycloalkyl;
optionally substituted alkenyl;
alkynyl;
optionally substituted haloalkyl;
optionally substituted heteroalkyl;
optionally substituted heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
optionally substituted alkoxy C₁-C₆ alkyl;
optionally substituted amino C₁-C₆ alkyl;
optionally substituted carboxy C₁-C₆ alkyl;
aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl;
optionally substituted aminocarbonyl C₃-C₈ cycloalkyl;
acylamino;
ureido; and
sulfonyl; or
wherein R¹⁵, R¹⁶ and R¹⁹ may further be selected from the following groups: 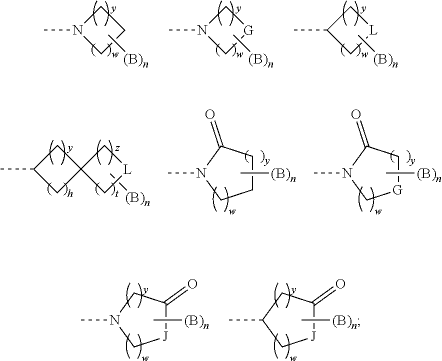
wherein y, w, z and t are independently:
an integer ranging from 1 to 3;
wherein h is:
an integer ranging from 0 to 3;
wherein n is:
an integer ranging from 0 to 4;
wherein G is selected from the list consisting of:
N—R²³;
O;
S; and
SO₂;
wherein J is selected from the list consisting of:
C(B)n; and
N—R²³,
wherein L is selected from the list consisting of:
C—(B)n;
N—R²³;
O;
S; and
SO₂;
wherein R²³ is selected from the list consisting of:
hydrogen;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted aryl;
optionally substituted heteroaryl;
C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
alkoxy C₁-C₆ alkyl;
optionally substituted amino C₁-C₆ alkyl;
optionally substituted carboxy C₁-C₆ alkyl;
optionally substituted aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl;
optionally substituted aminocarbonyl C₃-C₈ cycloalkyl; and
aminosulfonyl;
wherein B is selected from the group consisting of:
hydrogen;
halogen;
hydroxy;
CN;
CF₃;
CHF₂;
NH₂;
alkoxy;
amino;
carboxy;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
optionally substituted alkoxy C₁-C₆ alkyl;
optionally substituted amino C₁-C₆ alkyl;
optionally substituted carboxy C₁-C₆ alkyl;
aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl;
optionally substituted aminocarbonyl C₃-C₈ cycloalkyl;
acylamino;
ureido;
sulfonyl; and
sulfonylamino;
wherein R¹⁷ and R¹⁸ are independently selected from the list consisting of:
hydrogen;
halogen;
CN;
CF₃;
CHF₂;
alkoxy;
amino;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted C₃-C₈ cycloalkyl C₁-C₆ alkyl;
optionally substituted heterocycloalkyl C₁-C₆ alkyl;
optionally substituted alkoxy C₁-C₆ alkyl;
optionally substituted amino C₁-C₆ alkyl;
optionally substituted heterocycloalkyl C₃-C₈ cycloalkyl;
optionally substituted alkoxy C₃-C₈ cycloalkyl;
optionally substituted amino C₃-C₈ cycloalkyl;
acylamino;
ureido;
sulfonyl;
aminosulfonyl; and
sulfonylamino;
wherein R²⁰ and R²² are independently selected from the list consisting of:
hydrogen;
halogen;
CN;
CF₃;
CHF₂;
alkoxy;
amino;
optionally substituted C₁-C₆ alkyl;
optionally substituted C₃-C₈ cycloalkyl;
optionally substituted haloalkyl;
optionally substituted heteroalkyl;
optionally substituted heterocycloalkyl;
optionally substituted C₃-C₈ cycloalkyl C₁-C₆ alkyl;
optionally substituted heterocycloalkyl C₁-C₆ alkyl;
optionally substituted alkoxy C₁-C₆ alkyl;
optionally substituted amino C₁-C₆ alkyl;
optionally substituted heterocycloalkyl C₃-C₈ cycloalkyl;
optionally substituted alkoxy C₃-C₈ cycloalkyl;
and optionally substituted amino C₃-C₈ cycloalkyl;
wherein R²¹ is selected from the list consisting of:
hydrogen;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl aryl C₁-C₆ alkyl;
optionally substituted heteroaryl C₁-C₆ alkyl;
optionally substituted alkoxy C₁-C₆ alkyl;
optionally substituted amino C₁-C₆ alkyl;
optionally substituted carboxy C₁-C₆ alkyl;
optionally substituted aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl; and
optionally substituted aminocarbonyl C₃-C₈ cycloalkyl;
wherein R¹⁵ and R¹⁶ can be linked together to form an optionally substituted bicyclic heteroaryl having the following formula: 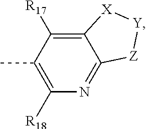
wherein X, Y and Z are each independently selected from the list consisting of: 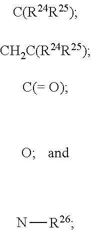
wherein R²⁴ and R²⁵ are each independently selected from the list consisting of:
hydrogen;
halogen;
hydroxy;
CN;
CF₃;
CHF₂;
NH₂;
alkoxy;
amino;
carboxy;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
optionally substituted alkoxy C₁-C₆ alkyl;
optionally substituted amino C₁-C₆ alkyl;
optionally substituted carboxy C₁-C₆ alkyl;
optionally substituted aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl;
optionally substituted aminocarbonyl C₃-C₈ cycloalkyl;
acylamino;
ureido;
sulfonyl; and
sulfonylamino;
wherein R²⁴ and R²⁵ can be linked together to form an optionally substituted C₃-C₈ cycloalkyl or an optionally substituted heterocycloalkyl;
wherein R²⁶ is selected from the group consisting of:
hydrogen;
aminocarbonyl;
alkoxy carbonyl;
optionally substituted C₁-C₆ alkyl;
optionally substituted C₃-C₈ cycloalkyl;
optionally substituted halo alkyl;
optionally substituted heteroalkyl;
optionally substituted heterocycloalkyl;
optionally substituted aryl;
optionally substituted heteroaryl;
optionally substituted C₃-C₈ cycloalkyl C₁-C₆ alkyl;
optionally substituted heterocycloalkyl C₁-C₆ alkyl;
optionally substituted aryl C₁-C₆ alkyl;
optionally substituted heteroaryl C₁-C₆ alkyl;
optionally substituted alkoxy C₁-C₆ alkyl;
amino C₁-C₆ alkyl;
optionally substituted carboxy C₁-C₆ alkyl;
optionally substituted aminocarbonyl C₁-C₆ alkyl;
optionally substituted heterocycloalkyl C₃-C₈ cycloalkyl;
optionally substituted aryl C₃-C₈ cycloalkyl;
optionally substituted heteroaryl C₃-C₈ cycloalkyl;
optionally substituted alkoxy C₃-C₈ cycloalkyl;
optionally substituted amino C₃-C₈ cycloalkyl;
optionally substituted carboxy C₃-C₈ cycloalkyl;
optionally substituted aminocarbonyl C₃-C₈ cycloalkyl; and
aminosulfonyl;
wherein R¹⁵ and R¹⁹ can be linked together to form an optionally substituted bicyclic heteroaryl;
wherein R¹⁵ and R²¹ can be linked together to form an optionally substituted bicyclic heteroaryl;
wherein R¹⁷ and R²¹ can be linked together to form an optionally substituted bicyclic heteroaryl;
wherein R²⁰ and R²¹ can be linked together to form an optionally substituted bicyclic heteroaryl;
as well as tautomers, geometrical isomers, optically active forms, and pharmaceutically acceptable salts thereof.

9. The method according to claim 20, wherein the NOX inhibitor is a NOX inhibitor according to Formula (V): 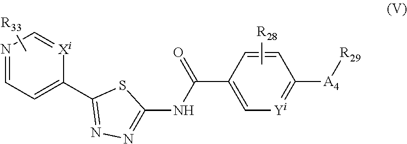
wherein:
Xⁱ is selected from the list consisting of CR²⁷ and N;
Yⁱ is selected from the list consisting of CH and N;
A₄ is selected from the list consisting of: 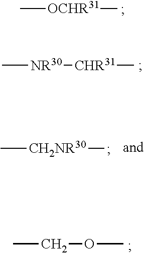
wherein R²⁷ is selected from the list consisting of:
H;
halogen;
and optionally substituted C₁-C₆ alkyl;
wherein R²⁸ is selected from the list consisting of:
H;
halogen (e.g. chloro, fluoro);
optionally substituted alkoxy such as optionally substituted methoxy(e.g. methoxy, (tetrahydro-2H-pyran-4-yl) methoxy, piperidin-4-ylmethoxy) or optionally substituted ethoxy (e.g. 2-(dimethylamino) ethoxy, 2-hydroxy ethoxy, 1-phenyl ethoxy, 2-methoxy ethoxy);
optionally substituted alkoxy C₁-C₆ alkyl;
optionally substituted C₁-C₆ alkyl such as optionally substituted methyl;
optionally substituted amino such as optionally substituted C₁-C₆ alkyl amino (e.g. methyl amino, tetrahydro-2H-pyran-4-yl)methyl)amino, (1-methylpiperidin-4-yl)methyl)amino, di-methyl amino, optionally substituted ethyl amino such as 2-morpholino ethyl amino or 2-(dimethylamino) ethyl amino or methoxy ethyl amino, optionally substituted methyl amino such as 1-methyl-1H-imidazol-4-yl methyl amino or 2-hydroxyethyl)amino, optionally substituted propyl amino such as dimethylamino propyl amino);
optionally substituted heterocycloalkyl such as optionally substituted piperazine (e.g. methylpiperazin-1-yl);
optionally substituted C₁-C₆ alkyl heterocycloalkyl such as optionally substituted C₁-C₆ alkyl piperazine (e.g. methylpiperazin-1-yl);
optionally substituted amino C₁-C₆ alkyl;
optionally substituted alkoxy C₁-C₆ alkyl;
—O—R³⁴; and
—NR³⁵R³⁶;
wherein R²⁹ is a group of formula —(CHR³²)n-A₅; or
R²⁹ forms with the moiety CHR³¹ from A₄ an optionally substituted ring selected from optionally substituted aryl such as an optionally substituted phenyl (e.g. phenyl or phenyl substituted by halogen such as fluoro phenyl substituted by alkoxy such as methoxy) and optionally substituted heteroaryl such as optionally substituted 1,3-dihydro-1H-indenyl (e.g. 1-(dimethylamino)-2,3-dihydro-1H-inden-2-yl, 2,3-dihydro-1H-inden-2-yl, 2,3-dihydro-1H-inden-1-yl) or optionally substituted 6,7-dihydro-5H-cyclopenta pyridinyl (e.g. 6,7-dihydro-5H-cyclopenta[b]pyridin-5-yl, 2-methylpyridin-3-yl, 5-methylpyridin-2-yl) or optionally substituted 1,2,3,4-tetrahydronaphthalenyl (e.g. 1,2,3,4-tetrahydronaphthalen-1-yl) or optionally substituted 2,3-dihydrobenzofuranyl (e.g. 2,3-dihydrobenzofuran-3-yl, 2,3-dihydro-1H-inden-1-yl) or optionally substituted thiadiazolyl (e.g. 1,3,4-thiadiazol-2-yl) or optionally substituted isoxazolyl (e.g. 5-methylisoxazol-3-yl) or optionally substituted pyrazolyl (e.g. 1-methyl-1H-pyrazol-3-yl) or optionally substituted imidazolyl (e.g. 1-methyl-1H-imidazol-2yl); or
R²⁹ forms with the moiety NR³⁰ from A₄ an optionally substituted ring selected from the list consisting of optionally substituted aryl and optionally substituted heteroaryl such as optionally substituted isoindolinyl (e.g. isoindolin-2-yl, 1H-indol-1-yl);
wherein n is an integer from 0 to 4 (such as 0, 1, 2, 3 or 4);
wherein R³⁰ is selected from H and optionally substituted alkyl, such as optionally substituted methyl;
wherein A₅ is an optionally substituted ring selected from optionally substituted aryl such as optionally substituted phenyl (e.g. methoxy phenyl, fluoro phenyl, chloro phenyl), optionally substituted heteroaryl such as optionally substituted pyridine (e.g. pyridin-2-yl, pyridin-3-yl, pyridin-4-yl, 2-methyl pyridin-3-yl, 5-methyl pyridin-2-yl) or optionally substituted pyrazolyl (e.g. 1,3-dimethyl-1H-pyrazol-5-yl, 1-methyl-1H-pyrazol-3-y) or optionally substituted thiadiazolyl (e.g. 1,3,4-thiadiazol-2-yl) or optionally substituted imidazolyl (e.g. 1H-imidazol-4-yl, 1-methyl-1H-imidazol-2-yl, 1-methyl-1H-imidazol-5-yl) or optionally substituted 1,2,4-triazolyl (e.g. 1-methyl-1H-1,2,4-triazol-5-yl) or optionally substituted isoxazolyl (e.g. 1-cyclopropylisoxazol-3-yl) or optionally substituted oxadiazolyl (e.g. 5-methyl-1,2,4-oxadiazol-3-yl) or optionally substituted pyrimidinyl (e.g. pyrimidinyl-2-yl);
wherein R³¹ is selected from the list consisting of:
H;
optionally substituted C₁-C₆ alkyl such as optionally substituted methyl (e.g. methoxy methyl, 3,3-difluoropyrrolidin-1-yl methyl, 4-methylpiperazin-1-yl methyl, hydroxyl methyl) or optionally substituted ethyl or optionally substituted propyl (e.g. methyl, hydroxy methyl, hydroxy ethyl, 2-propanolyl, hydroxyl isopropyl);
optionally substituted amino C₁-C₆ alkyl such as optionally substituted amino methyl (e.g. dimethylamino methyl, methylamino methyl);
optionally substituted alkoxy C₁-C₆ alkyl;
optionally substituted heterocycloalkyl C₁-C₆ alkyl such as optionally substituted heterocycloalkyl methyl for example optionally substituted pyrrolidine C₁-C₆ alkyl (e.g. 3,3-difluoropyrrolidin-1-yl methyl) or substituted piperazine C₁-C₆ alkyl (e.g. 4-methylpiperazin-1-yl methyl) or heterocycloalkyl ethyl for example optionally substituted morpholino C₁-C₆ alkyl (e.g. morpholino methyl, morpholino ethyl) or optionally substituted pyrrolidine C₁-C₆ alkyl (e.g. pyrrolidine methyl, pyrrolidine ethyl);
optionally substituted aminocarbonyl (e.g. dimethyl aminocarbonyl);
optionally substituted C₂-C₈ cycloalkyl such as optionally substituted cyclopropyl; and
optionally substituted amino C₁-C₆ alkyl such as optionally substituted amino ethyl (e.g. di-methyl amino ethyl) or optionally substituted amino methyl (e.g. di-methyl amino methyl);
wherein R³² is selected from the list consisting of:
H;
optionally substituted C₁-C₆ alkyl such as optionally substituted methyl;
optionally substituted amino;
optionally substituted C₁-C₆ alkyl amino (e.g. dimethyl amino); and
hydroxy; and
wherein R³² groups are independently selected for each repeating unit (CHR³²);
wherein R³³ is selected from the list consisting of:
H;
halogen (e.g. fluoro); and
optionally substituted C₁-C₆ alkyl such as methyl;
wherein R³⁴ is selected from the group consisting of:
H;
optionally substituted C₁-C₆ alkyl such as optionally substituted methyl or optionally substituted ethyl (e.g. methoxy ethyl, 2-(dimethylamino)ethyl, hydroxy ethyl);
optionally substituted amino C₁-C₆ alkyl;
optionally substituted heterocycloalkyl;
optionally substituted C₂-C₈ cycloalkyl;
optionally substituted heterocycloalkyl C₁-C₆ alkyl such as optionally substituted heterocycloalkyl methyl, for example optionally substituted tetrahydropyran C₁-C₆ alkyl (e.g. tetrahydro-2H-pyran-4-yl) or optionally substituted piperidine alkyl (e.g. 1-methylpiperidin-4-yl);
optionally substituted C₂-C₈ cycloalkyl C₁-C₆ alkyl;
optionally substituted alkoxy;
optionally substituted amino C₁-C₆ alkyl such optionally substituted amino ethyl (e.g. 2-(dimethylamino)ethyl);
optionally substituted aryl C₁-C₆ alkyl; and
optionally substituted heteroaryl C₁-C₆ alkyl;
wherein R³⁵ and R³⁶ are independently selected from the list consisting of:
H;
optionally substituted C₁-C₆ alkyl such as optionally substituted methyl (e.g. 1-methyl-1H-imidazol-4-yl)methyl)) or optionally substituted ethyl (e.g. 2-methoxy ethyl);
optionally substituted amino C₁-C₆ alkyl such as optionally substituted amino ethyl (e.g. dimethyl amino ethyl) or such as optionally substituted amino propyl (e.g. dimethylamino)propyl);
optionally substituted heterocycloalkyl such as optionally substituted piperidine (e.g. 1-methylpiperidin);
optionally substituted C₂-C₈ cycloalkyl;
optionally substituted heterocycloalkyl C₁-C₆ alkyl such as optionally substituted heterocycloalkyl ethyl for example optionally substituted morpholino C₁-C₆ alkyl (e.g. 2-morpholino ethyl) or optionally substituted heterocycloalkyl methyl for example optionally substituted tetrahydrofuran C₁-C₆ alkyl (e.g. tetrahydro-2H-pyran-4-yl methyl) or piperidine C₁-C₆ alkyl (e.g. 1-methylpiperidin-4-yl) methyl or optionally substituted imidazolyl C₁-C₆ alkyl (e.g. 1-methyl-1H-imidazol-4-yl)methyl) optionally substituted C₂-C₈ cycloalkyl C₁-C₆ alkyl;
optionally substituted alkoxy;
optionally substituted alkoxy C₁-C₆ alkyl such as optionally substituted alkoxy ethyl (e.g. 2-methoxy ethyl);
optionally substituted aryl C₁-C₆ alkyl; and
optionally substituted heteroaryl C₁-C₆ alkyl such as heteroaryl C₁-C₆ alkyl methyl, for example optionally substituted imidazolyl C₁-C₆ alkyl (e.g. 1-methyl-1H-imidazol-4-yl methyl), optionally substituted amino C₁-C₆ alkyl such optionally substituted amino ethyl or optionally substituted amino propyl (e.g. 2-(dimethylamino)ethyl, 2-(dimethylamino)propyl)); as well as tautomers, geometrical isomers, optically active forms, pharmaceutically acceptable salts and pharmaceutically active derivative thereof.

10. The method according to claim 20, wherein the NOX inhibitor is selected from the group consisting of:
2-(2-chlorophenyl)-4-methyl-5-(pyridin-2-ylmethyl)-1H-pyrazolo[4,3-c]pyridine-3,6(2H,5H)-dione;
2-(2-chlorophenyl)-4-[3-(dimethylamino)phenyl]-5-methyl-1H-pyrazolo[4,3-c]pyridine-3,6(2H,5H)-dione;
4-(2-fluoro-4-methoxyphenyl)-2-(2-methoxyphenyl)-5-(pyridin-3-ylmethyl)-1H-pyrazolo[4,3-c]pyridine-3,6(2H,5H)-dione;
(R)-3-methoxy-4-(2-morpholino-1-phenylethoxy)-N-(5-(pyridin-4-yl)-1,3,4-thiadiazol-2-yl)benzamide;
10-benzyl-2-(2-chlorophenyl)-2,3,8,9,10,11-hexahydro-1H-pyrazolo[4′,3′:3,4]pyrido[1,2-a][1,4]diazepine-1,5(7H)-dione;
(S)-3-methoxy-4-(1-phenylethoxy)-N-(5-(pyridin-4-yl)-1,3,4-thiadiazol-2-yl)benzamide;
(R)-4-(2-hydroxy-1-phenylethoxy)-3-methoxy-N-(5-(pyridin-4-yl)-1,3,4-thiadiazol-2-yl)benzamide; and
(R)-4-(2-(dimethylamino)-1-phenylethoxy)-3-methoxy-N-(5-(pyridin-4-yl)-1,3,4-thiadiazol-2-yl)benzamide.

11. The method according to claim 8, wherein the NOX inhibitor is selected from the group consisting of:
5-(2-methoxypyridin-4-yl)-2-(2-methylpyridin-4-yl)-1H-indole;
2,5-bis(2-methylpyridin-4-yl)-1H-indole;
4-(2-(2-methylpyridin-4-yl)-1H-indol-5-yl)pyridin-2-amine;
5-(5-fluoropyridin-3-yl)-2-(2-methylpyridin-4-yl)-1H-indole;
5-(5-chloropyridin-3-yl)-2-(2-methylpyridin-4-yl)-1H-indole;
5-(5-isopropoxypyridin-3-yl)-2-(2-methylpyridin-4-yl)-1H-indole;
N,N-dimethyl-5-(2-(2-methylpyridin-4-yl)-1H-indol-5-yl)pyridin-3-amine;
5-(6-methylpyridin-3-yl)-2-(2-methylpyridin-4-yl)-1H-indole;
2-(2-methylpyridin-4-yl)-5-(pyrimidin-5-yl)-1H-indole;
2-methyl-5-(2-(2-methylpyridin-4-yl)-1H-indol-5-yl)-1H-benzo[d]imidazole;
2-(2-(azetidin-1-yl)pyridin-4-yl)-5-(5-methoxypyridin-3-yl)-1H-indole;
5-(5-methoxypyridin-3-yl)-2-(2-methoxypyridin-4-yl)-1H-indole;
2-(2,6-dimethylpyridin-4-yl)-5-(5-methoxypyridin-3-yl)-1H-indole;
5-(5-methoxypyridin-3-yl)-2-(2-methylpyrimidin-4-yl)-1H-indole;
5-(5-methoxypyridin-3-yl)-2-(1-methyl-1H-imidazol-5-yl)-1H-indole;
4-(5-(5-methoxypyridin-3-yl)-1H-indol-2-yl)-2-methylthiazole;
2-(2-cyclopropylpyridin-4-yl)-5-(5-methoxypyridin-3-yl)-1H-indole;
1-(4-(5-(5-methoxypyridin-3-yl)-1H-indol-2-yl)pyridin-2-yl)azetidin-3-ol;
1-(4-(5-(5-methoxypyridin-3-yl)-1H-indol-2-yl)pyridin-2-yl)-6-oxa-1-azaspiro[3.3]heptane;
2-(2-(4,4-difluoropiperidin-1-yl)pyridin-4-yl)-5-(5-methoxypyridin-3-yl)-1H-indole;
4-(4-(5-(5-methoxypyridin-3-yl)-1H-indol-2-yl)pyridin-2-yl)morpholine;
2-(5-fluoropyridin-3-yl)-5-(5-methoxypyridin-3-yl)-1H-indole;
5-(5-methoxypyridin-3-yl)-2-(6-methoxypyridin-3-yl)-1H-indole;
4-(5-(5-(5-methoxypyridin-3-yl)-1H-indol-2-yl)pyridin-2-yl)morpholine;
5-(5-methoxypyridin-3-yl)-2-(6-(oxetan-3-yl)pyridin-3-yl)-1H-indole;
1-(5-(5-(5-methoxypyridin-3-yl)-1H-indol-2-yl)pyridin-2-yl)-6-oxa-1-azaspiro[3.3]heptane;
N,N-dimethyl-5-(5-(2-methylpyridin-4-yl)-1H-indol-2-yl)pyridin-2-amine;
4-(5-(5-methoxypyridin-3-yl)-1H-indol-2-yl)-N,N-dimethylpyridin-2-amine;
N,N-dimethyl-4-(5-(quinolin-4-yl)-1H-indol-2-yl)pyridin-2-amine;
1-(5-(6-chloro-5-(5-methoxypyridin-3-yl)-1H-indol-2-yl)pyridin-2-yl)-6-oxa-1-azaspiro[3.3]heptane;
1-(4-(6-chloro-5-(5-methoxypyridin-3-yl)-1H-indol-2-yl)pyridin-2-yl)-6-oxa-1-azaspiro[3.3]heptane;
1-(4-(6-chloro-5-(2-methylpyridin-4-yl)-1H-indol-2-yl)pyridin-2-yl)-6-oxa-1-azaspiro[3.3]heptane;
6-(5-(6-chloro-5-(5-methoxypyridin-3-yl)-1H-indol-2-yl)pyridin-2-yl)-2-oxa-6-azaspiro[3.3]heptane;
2-(5-(6-chloro-5-(5-methoxypyridin-3-yl)-1H-indol-2-yl)pyridin-2-yl)-7-oxa-2-azaspiro[3.5]nonane;
1-(5-(6-chloro-5-(5-methoxypyridin-3-yl)-1H-indol-2-yl)pyridin-2-yl)-3-methylazetidin-3-ol;
6-chloro-2-(6-(3,3-difluoroazetidin-1-yl)pyridin-3-yl)-5-(5-methoxypyridin-3-yl)-1H-indole;
1-(5-(6-chloro-5-(5-methoxypyridin-3-yl)-1H-indol-2-yl)pyridin-2-yl)azetidine-3-carboxylic acid;
1-(5-(6-chloro-5-(5-methoxypyridin-3-yl)-1H-indol-2-yl)pyridin-2-yl)azetidine-3-carboxamide;
(1-(5-(6-chloro-5-(5-methoxypyridin-3-yl)-1H-indol-2-yl)pyridin-2-yl)azetidin-3-yl) methanol;
6-chloro-5-(5-methoxypyridin-3-yl)-2-(6-(3-methoxypyrrolidin-1-yl)pyridin-3-yl)-1H-indole;
N-(1-(5-(6-chloro-5-(5-methoxypyridin-3-yl)-1H-indol-2-yl)pyridin-2-yl)pyrrolidin-3-yl)methanesulfonamide;
(1-(5-(6-chloro-5-(5-methoxypyridin-3-yl)-1H-indol-2-yl)pyridin-2-yl)pyrrolidin-2-yl) methanol;
1-(5-(6-chloro-5-(5-methoxypyridin-3-yl)-1H-indol-2-yl)pyridin-2-yl)-5-(hydroxymethyl)pyrrolidin-2-one;
4-(5-(6-chloro-5-(5-methoxypyridin-3-yl)-1H-indol-2-yl)-3-fluoropyridin-2-yl) morpholine;
6-chloro-5-(5-methoxypyridin-3-yl)-2-(6-(4-(oxetan-3-yl) piperazin-1-yl)pyridin-3-yl)-1H-indole;
6-chloro-2-(6-(4-ethylpiperazin-1-yl)pyridin-3-yl)-5-(5-methoxypyridin-3-yl)-1H-indole 4-(5-(6-chloro-5-(5-methoxypyridin-3-yl)-1H-indol-2-yl)pyridin-2-yl)thiomorpholine 1,1-dioxide;
6-chloro-2-(6-(4,4-difluoropiperidin-1-yl)pyridin-3-yl)-5-(5-methoxypyridin-3-yl)-1H-indole;
4-(5-(6-chloro-5-(5-methoxypyridin-3-yl)-1H-indol-2-yl)pyridin-2-yl)-1-methylpiperazin-2-one;
3′-(6-chloro-5-(5-methoxypyridin-3-yl)-1H-indol-2-yl)-1-methyl-5′H-spiro[azetidine-3,7′-furo[3,4-b]pyridine];
7-(6-chloro-5-(5-methoxypyridin-3-yl)-1H-indol-2-yl)-4-methyl-3,4-dihydro-2H-pyrido[3,2-b][1,4]oxazine;
(5-(6-chloro-5-(5-methoxypyridin-3-yl)-1H-indol-2-yl)pyridin-2-yl) (oxetan-3-yl) methanol;
6-chloro-2-(6-(methoxy(oxetan-3-yl)methyl)pyridin-3-yl)-5-(5-methoxypyridin-3-yl)-1H-indole;
2-(5-(6-chloro-5-(5-methoxypyridin-3-yl)-1H-indol-2-yl)pyridin-2-yl) propan-1-ol;
5-(6-chloro-5-(5-methoxypyridin-3-yl)-1H-indol-2-yl)-1-methylpyridin-2(1H)-one;
4-(6-chloro-5-(5-methoxypyridin-3-yl)-1H-indol-2-yl)-1-cyclopropylmethyl)pyridin-2(1H)-one;
4-(5-(7-chloro-5-(5-methoxypyridin-3-yl)-1H-indol-2-yl)pyridin-2-yl)morpholine;
4-(5-(4,6-difluoro-5-(5-methoxypyridin-3-yl)-1H-indol-2-yl)pyridin-2-yl)morpholine;
4-(5-(6-chloro-5-(quinolin-5-yl)-1H-indol-2-yl)pyridin-2-yl)morpholine;
4-(5-(6-chloro-5-(5-chloropyridin-3-yl)-1H-indol-2-yl)pyridin-2-yl)morpholine;
4-fluoro-5-(5-methoxypyridin-3-yl)-2-(2-methylpyridin-4-yl)-1H-indole;
6-fluoro-5-(5-methoxypyridin-3-yl)-2-(2-methylpyridin-4-yl)-1H-indole;
4-chloro-5-(5-methoxypyridin-3-yl)-2-(2-methylpyridin-4-yl)-1H-indole;
6-chloro-5-(5-methoxypyridin-3-yl)-2-(2-methylpyridin-4-yl)-1H-indole;
5-(5-methoxypyridin-3-yl)-4-methyl-2-(2-methylpyridin-4-yl)-1H-indole;
5-(5-methoxypyridin-3-yl)-6-methyl-2-(2-methylpyridin-4-yl)-1H-indole;
4-methyl-2,5-di(pyridin-4-yl)-1H-indole;
6-methyl-2,5-di(pyridin-4-yl)-1H-indole;
4-(5-(4-chloro-5-(5-methoxypyridin-3-yl)-1H-indol-2-yl)pyridin-2-yl)morpholine;
4-(5-(6-chloro-5-(5-methoxypyridin-3-yl)-1H-indol-2-yl)pyridin-2-yl)morpholine;
4-chloro-2,5-di(pyridin-4-yl)-1H-indole 6-chloro-2,5-di(pyridin-4-yl)-1H-indole;
N,N-dimethyl-4-(2-(2-methylpyridin-4-yl)-1H-indol-5-yl)pyridin-2-amine;
5-(6-cyclopropylpyridin-3-yl)-2-(2-methylpyridin-4-yl)-1H-indole;
5-(6-methoxypyridin-3-yl)-2-(2-methylpyridin-4-yl)-1H-indole;
5-(6-(methoxymethyl)pyridin-3-yl)-2-(2-methylpyridin-4-yl)-1H-indole;
5-(5-ethoxypyridin-3-yl)-2-(2-methylpyridin-4-yl)-1H-indole;
5-(5-cyclopropoxypyridin-3-yl)-2-(2-methylpyridin-4-yl)-1H-indole;
5-(5-(cyclopropylmethoxy)pyridin-3-yl)-2-(2-methylpyridin-4-yl)-1H-indole;
5-(5-cyclopropylpyridin-3-yl)-2-(2-methylpyridin-4-yl)-1H-indole;
7-(2-(2-methylpyridin-4-yl)-1H-indol-5-yl)-3,4-dihydro-2H-pyrido[3,2-b][1,4]oxazine;
3-(2-(2-methylpyridin-4-yl)-1H-indol-5-yl)quinoline;
N-methyl-4-(2-(2-methylpyridin-4-yl)-1H-indol-5-yl)pyridin-2-amine;
4-(4-(2-(2-methylpyridin-4-yl)-1H-indol-5-yl)pyridin-2-yl)morpholine;
5-(6-chloro-2-(6-morpholinopyridin-3-yl)-1H-indol-5-yl) quinolin-2-ol;
4-(5-(6-chloro-5-(thieno[2,3-c]pyridin-4-yl)-1H-indol-2-yl)pyridin-2-yl)morpholine;
4-(5-(6-chloro-5-(6-fluoroquinolin-4-yl)-1H-indol-2-yl)pyridin-2-yl)morpholine;
4-(5-(6-chloro-5-(2-methoxyquinolin-5-yl)-1H-indol-2-yl)pyridin-2-yl)morpholine; 4-(5-(6-fluoroquinolin-4-yl)-1H-indol-2-yl)-N,N-dimethylpyridin-2-amine;
4-(5-(isoquinolin-4-yl)-1H-indol-2-yl)-N,N-dimethylpyridin-2-amine;
N,N-dimethyl-4-(5-(quinolin-5-yl)-1H-indol-2-yl)pyridin-2-amine;
3-chloro-2,5-di(pyridin-4-yl)-1H-indole;
2-(2-methylpyridin-4-yl)-5-(pyridin-4-yl)-1H-indole;
5-(5-methoxypyridin-3-yl)-2-(2-methylpyridin-4-yl)-1H-indole;
5-(5-methoxypyridin-3-yl)-2-(pyridin-4-yl)-1H-indole;
5-(5-methoxypyridin-3-yl)-2-(pyridin-3-yl)-1H-indole;
4-(2-(pyridin-4-yl)-1H-indol-5-yl)quinoline;
4-(2-(pyridin-3-yl)-1H-indol-5-yl)quinoline;
2,5-di(pyridin-4-yl)-1H-indole;
2-(pyridin-3-yl)-5-(pyridin-4-yl)-1H-indole;
2,5-di(pyridin-3-yl)-1H-indole;
2-(1-methyl-1H-pyrazol-5-yl)-5-(pyridin-4-yl)-1H-indole;
5-(2-(4-methylpiperazin-1-yl)pyridin-4-yl)-2-(pyridin-4-yl)-1H-indole;
5-(5-methoxypyridin-3-yl)-2-(pyridin-2-yl)-1H-indole;
5-(5-methoxypyridin-3-yl)-2-(pyrimidin-4-yl)-1H-indole;
2-(3-fluoropyridin-4-yl)-5-(5-methoxypyridin-3-yl)-1H-indole;
1-isopropyl-5-(5-methoxypyridin-3-yl)-2-(2-methylpyridin-4-yl)-1H-indole;
4-(5-(6-chloro-5-(5-methoxypyridin-3-yl)-1-methyl-1H-indol-2-yl)pyridin-2-yl) morpholine;
1-(2-methoxyethyl)-2,5-di(pyridin-4-yl)-1H-indole;
1-(2-methoxyethyl)-5-(5-methoxypyridin-3-yl)-2-(2-methylpyridin-4-yl)-1H-indole;
1-methyl-5-(5-methoxypyridin-3-yl)-2-(2-methylpyridin-4-yl)-1H-indole;
1-ethyl-5-(5-methoxypyridin-3-yl)-2-(2-methylpyridin-4-yl)-1H-indole;
1-(cyclopropylmethyl)-5-(5-methoxypyridin-3-yl)-2-(2-methylpyridin-4-yl)-1H-indole;
1-(benzyl)-5-(5-methoxypyridin-3-yl)-2-(2-methylpyridin-4-yl)-1H-indole;
7-(4-chloro-5-(5-methoxypyridin-3-yl)-1-methyl-1H-indol-2-yl)-4-methyl-3,4-dihydro-2H-pyrido[3,2-b][1,4]oxazine;
7-(6-chloro-5-(5-methoxypyridin-3-yl)-1-methyl-1H-indol-2-yl)-4-methyl-3,4-dihydro-2H-pyrido[3,2-b][1,4]oxazine;
1-cyclopropyl-5-(5-methoxypyridin-3-yl)-2-(2-methylpyridin-4-yl)-1H-indole;
1-methyl-2,5-di(pyridin-4-yl)-1H-indole; and
5-(5-methoxypyridin-3-yl)-2-(3-methylpyridin-4-yl)-1H-indole.

12. The method according to claim 20, wherein the NOX inhibitor is 2-(2-chlorophenyl)-4-[3-(dimethylamino)phenyl]-5-methyl-1H-pyrazolo[4,3-c]pyridine-3,6(2H,5H)-dione.

13. The method according to claim 20, wherein the NOX inhibitor, or a pharmaceutically-acceptable salt thereof, is administered in the form of a combination product comprising:
(a) the NOX inhibitor, or a pharmaceutically-acceptable salt thereof; and
(b) an ACE inhibitor, or a pharmaceutically-acceptable salt thereof, and/or an ARB, or a pharmaceutically-acceptable salt thereof.

14. The method according to claim 13, wherein the combination product comprises a pharmaceutical formulation including the NOX inhibitor; the ACE inhibitor and/or the ARB; and a pharmaceutically-acceptable adjuvant, diluent or carrier.

15. The method according to claim 14, wherein the ACE inhibitor is selected from the list consisting of ramipril, benazepril, captopril, enalapril, fosinopril, lisinopril, moexipril, perindopril, quinapril, and trandolapril.

16. The method according to claim 13, wherein the combination product comprises:
(a) 2-(2-chlorophenyl)-4-[3-(dimethylamino)phenyl]-5-methyl-1H-pyrazolo[4,3-c]pyridine-3,6(2H,5H)-dione, or a pharmaceutically-acceptable salt thereof; and
(b) ramipril, or a pharmaceutically-acceptable salt thereof.

17. The method according to claim 13, wherein the ARB is present and is selected from the list consisting of azilsartan, candesartan, eprosartan, sparsentan, irbesartan, losartan, Olmesartan, telmisartan and valsartan.

18. A combination product which comprises a kit of parts comprising components:
(A) a pharmaceutical formulation including a NOX inhibitor, or a pharmaceutically-acceptable salt thereof, in admixture with a pharmaceutically-acceptable adjuvant, diluent or carrier; and
(B) a pharmaceutical formulation included an ACE inhibitor, or a pharmaceutically-acceptable salt thereof, and/or an ARB, or a pharmaceutically-acceptable salt thereof, in admixture with a pharmaceutically-acceptable adjuvant, diluent or carrier,
which components (A) and (B) are each provided in a form that is suitable for administration in conjunction with the other.

19. A kit of parts comprising:
(I) one of components (A) and (B)
(A) a pharmaceutical formulation including a NOX inhibitor, or a pharmaceutically-acceptable salt thereof, in admixture with a pharmaceutically-acceptable adjuvant, diluent or carrier; and
(B) a pharmaceutical formulation included an ACE inhibitor, or a pharmaceutically-acceptable salt thereof, and/or an ARB, or a pharmaceutically-acceptable salt thereof, in admixture with a pharmaceutically-acceptable adjuvant, diluent or carrier;
together with
(II) instructions to use the one component in conjunction with the other of the two components.

20. A method of treatment of Alport syndrome, which method comprises:
administering a NOX inhibitor, or a pharmaceutically acceptable salt thereof, to a patient in need of such treatment.

21. (canceled)
